(19) United States
(12) Patent Application Publication
(10) Pub. No.: US 2025/0345348 A1
(43) Pub. Date: Nov. 13, 2025

(54) AMPK ACTIVATOR (CBDA) AND SGLT2 INHIBITOR FOR METABOLIC HEALTH

(71) Applicant: SOCIETE DES PRODUITS NESTLE S.A., Vevey (CH)

(72) Inventors: MATTHEW SANDERS, Epalinges (CH); JOY RICHARD, Corcelles Le Jorat (CH); YANN RATINAUD, Morges (CH); ROBIN WILLOWS, Echandens (CH)

(21) Appl. No.: 18/862,421

(22) PCT Filed: Apr. 28, 2023
(86) PCT No.: PCT/EP2023/061283

§ 371 Date: Nov. 1, 2024

Related U.S. Application Data
(60) Provisional application No. 63/338,197, filed on May 4, 2022.

(51) Int. Cl. A61K 31/00; A61K 31/351; A61P 3/08

(52) U.S. Cl. CPC A61K 31/658; A61K 31/351; A61P 3/08

(57) ABSTRACT

A composition includes a first compound having general formula I and a second compound for use in the synergistic activation of AMPK. A method of using the composition in the synergistic activation of AMPK is also provided.

### BACKGROUND

The present disclosure generally relates to compositions and methods for improving metabolic health. Specifically, the present disclosure relates to a combination of an AMP-activated protein kinase (AMPK) activator (e.g., CBDA, CBGA or CBNA) and a sodium-glucose cotransporter-2 (SGLT2) inhibitor for improving metabolic health. For example, a combination of an AMP-activated protein kinase (AMPK) activator (e.g., CBDA, CBGA or CBNA) and a SGLT2 inhibitor for synergistically activate AMPK and improving metabolic health.

AMP-activated protein kinase (AMPK) is an evolutionarily conserved master regulator of energy homeostasis that coordinates metabolic pathways in order to balance nutrient supply with energy demand. AMPK is considered a key drug target to combat the growing epidemic of metabolic disorders such as obesity, type 2 diabetes, cardiovascular disease.

AMPK activity is found in all tissues, including liver, kidney, muscle, lung, and brain (PMID: 10698692). In terms of structure, AMPK is a heterotrimeric complex consisting of a catalytic subunit (α) and two regulatory subunits (β and γ). The AMPK complex is evolutionarily conserved and also can be found in yeast and plants. Mammalian AMPK is composed of different isoforms of subunits: α1, α2, β1, β2, γ1, γ2, and γ3 (PMID: 11746230) leading to 12 possible heterotrimeric combinations. The α2 isoform is predominately found in skeletal and cardiac muscle AMPK; both the α1 and α2 isoforms are found in hepatic AMPK; while for example in adipose and T cells the al isoform AMPK predominates (PMID: 16818670, PMID 15878856).

There is no direct AMPK-activating drug available to treat metabolic disorders despite intensive efforts continuously made by the pharmaceutical industry. Several synthetic AMPK activators have been identified/developed. However, they either have no/poor oral availability (PMID: 16753576, PMID: 24900234) or there are concerns about their off-target, adverse effects, since chronic and strong AMPK activation may cause increases in cardiac glycogen content and hypertrophy (PMID: 11827995).

Finding natural bioactive molecules that moderately activate AMPK especially in muscle, liver and kidney with defined mechanism of action are likely to provide exercise-mimetic effects and help maintain/improve metabolic health.

There are numerous natural compounds/extracts known to bring about some metabolic health benefits that are shown to indirectly stimulate AMPK most likely through inhibition of mitochondrial respiration. However, whether those metabolic effects are mediated by AMPK is largely elusive, and moreover there are concerns regarding side/toxic effects such as cellular or mitochondrial poisoning.

Sodium-glucose cotransporter-2 (SGLT2) inhibitors are an insulin-independent class of oral antihyperglycemic medication that clinicians use in the treatment of type 2 diabetes. There are four SGLT2 inhibitors approved by the Food and Drug Administration since 2013: canagliflozin, dapagliflozin, empagliflozin and ertugliflozin.

SGLT2 inhibitors are a class of prescription medicines that are FDA-approved for use with diet and exercise to lower blood sugar in adults with type 2 diabetes. Medicines in the SGLT2 inhibitor class include canagliflozin, dapagliflozin, and empagliflozin. They are available as single-ingredient products and also in combination with other diabetes medicines such as metformin. SGLT2 inhibitors lower blood sugar by causing the kidneys to remove sugar from the body through the urine.

There is a clear unmet need for new compositions a combination of an AMP-activated protein kinase (AMPK) activator (e.g., CBDA, CBGA or CBNA) and a sodium-glucose cotransporter-2 (SGLT2) inhibitor for improving metabolic health. Needed in the art is the combination of an AMP-activated protein kinase (AMPK) activator (e.g., CBDA, CBGA or CBNA) and a sodium-glucose cotransporter-2 (SGLT2) inhibitor for synergistically activating AMPK, thus improving metabolic health.

### SUMMARY

The present disclosure includes the recognition that administering a subject a combination of an AMP-activated protein kinase (AMPK) activator (e.g., CBDA, CBGA or CBNA) and a sodium-glucose cotransporter-2 (SGLT2) inhibitor for synergistically activating AMPK would represent a breakthrough for improving metabolic health. Indeed, the method of administering a subject with a combination of an AMP-activated protein kinase (AMPK) activator (e.g., CBDA, CBGA or CBNA) and a sodium-glucose cotransporter-2 (SGLT2) inhibitor disclosed herein, led to improved performance of activated AMPK as compared to administering an AMP-activated protein kinase (AMPK) activator (e.g., CBDA, CBGA or CBNA) or a sodium-glucose cotransporter-2 (SGLT2) inhibitor alone.

### BRIEF DESCRIPTION OF DRAWINGS

FIG. 1 (FIG. 1A and FIG. 1B) is a set of graphs showing glucose clearance in Zebrafish according to certain embodiments of the present invention. FIG. 1A shows that a combination of CBD and Dapa (a SGLT-2 inhibitor) improves the glucose clearance of Zebrafish as compared with CBD and Dapa alone. FIG. 1B shows that a combination of CBDA and Dapa (an SGLT-2 inhibitor) improves the glucose clearance of Zebrafish as compared with CBDA and Dapa alone.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one dithionite or a functionally similar reducing agent” should be interpreted as “dithionite,” or “a functionally similar reducing agent,” or “both dithionite and a functionally similar reducing agent.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The term “cannabidiolic acid,” or “CBDA,” as used herein, refers to 2,4-dihydroxy-3-[(1R,6R)-3-methyl-6-prop-1-en-2-ylcyclohex-2-en-1-yl]-6-pentylbenzoic acid, with CAS number of 1244-58-2. It is also called benzoic acid, 2,4-dihydroxy-3-[(1R,6R)-3-methyl-6-(1-methylethenyl)-2-cyclohexen-1-yl]-6-pentyl-, benzoic acid, 2,4-dihydroxy-3-[3-methyl-6-(1-methylethenyl)-2-cyclohexen-1-yl]-6-pentyl-, (1R-trans)-, β-Resorcylic acid, 3-p-mentha-1,8-dien-3-yl-6-pentyl-, Cannabidiolcarboxylic acid or Cannabinoid CBDA. It has the formula as follows:

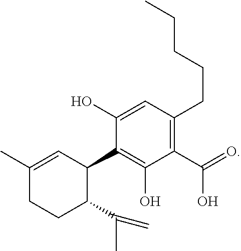 [SMILES: C=C(C)[C@@H]1CCC(C)=C[C@H]1c1c(O)cc(CCCCC)c(C(=O)O)c1O]

The term “resorcylic acid,” as used herein, refers to a type of dihydroxybenzoic acid. In one embodiment, a resorcylic acid may include 3,5-Dihydroxybenzoic acid (α-Resorcylic acid), 2,4-Dihydroxybenzoic acid (β-Resorcylic acid) or 2,6-Dihydroxybenzoic acid (γ-Resorcylic acid) or any of their derivatives.

The term “cannabidiol,” or “CBD,” as used herein, refers to 2-[(1R,6R)-6-Isopropenyl-3-methylcyclohex-2-en-1-yl]-5-pentylbenzene-1,3-diol. It is also called 1,3-Benzenediol, 2-[(1R,6R)-3-methyl-6-(1-methylethenyl)-2-cyclohexen-1-yl]-5-pentyl-, Resorcinol, 2-p-mentha-1,8-dien-3-yl-5-pentyl-, trans-(−)-, 1,3-Benzenediol, 2-[3-methyl-6-(1-methylethenyl)-2-cyclohexen-1-yl]-5-pentyl-, (1R-trans)-, Epidiolex, GWP 42003P or Epidyolex. It has the formula as follows:

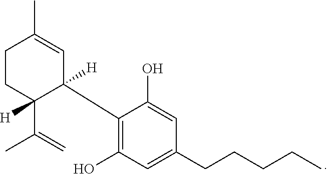 [SMILES: C=C(C)[C@@H]1CCC(C)=C[C@H]1c1c(O)cc(CCCCC)cc1O]

The term “cannabigerolic acid,” or “CBGA,” as used herein, refers to the acidic form of cannabigerol (CBG). It is a dihydroxybenzoic acid and olivetolic acid in which the hydrogen at position 3 is substituted by a geranyl group. It is a biosynthetic precursor to Delta-9-tetrahydrocannabinol, which is the principal psychoactive constituent of the Cannabis plant. It is also a diterpenoid, a polyketide, a member of resorcinols and a phytocannabinoid. It derives from an olivetolic acid. It is a conjugate acid of a cannabigerolate. It has the formula as follows:

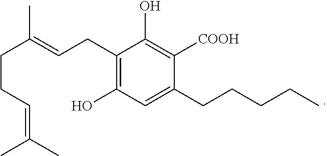 [SMILES: CCCCCc1cc(O)c(C/C=C(\C)CCC=C(C)C)c(O)c1C(=O)O]

The term “cannabinolic acid,” or “CBNA,” as used herein, refers to 1-Hydroxy-6,6,9-trimethyl-3-pentyl-6H-benzo[c]chromene-2-carboxylic acid. It is also called 6H-Dibenzo[b,d]pyran-2-carboxylic acid, 1-hydroxy-6,6,9-trimethyl-3-pentyl-, 1-hydroxy-6,6,9-trimethyl-3-pentylbenzo[c]chromene-2-carboxylic acid or 6H-Dibenzo(b,d)pyran-2-carboxylic acid, 1-hydroxy-6,6,9-trimethyl-3-pentyl-. It has the formula as follows:

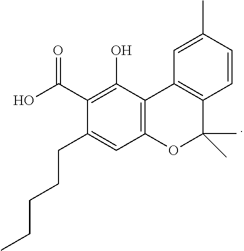 [SMILES: CCCCCc1cc2c(c(O)c1C(=O)O)-c1cc(C)ccc1C(C)(C)O2]

In one embodiment, the first compound of the present invention comprises CBNA or its derivatives or analogues. WO2021/105075 discloses examples of CBNA derivatives or analogues.

The term “5′-adenosine monophosphate-activated protein kinase,” “AMP-activated protein kinase,” or “AMPK,” as used herein, refers to a heterotrimeric kinase composed of an alpha catalytic subunit, and non-catalytic beta and gamma subunits. AMPK is an important energy-sensing enzyme that monitors cellular energy status. In response to cellular metabolic stresses, AMPK is activated and phosphorylates and inactivates acetyl-CoA carboxylase (ACC) and beta-hydroxy beta-methylglutaryl-CoA reductase (HMGCR), key enzymes involved in regulating de novo biosynthesis of fatty acid and cholesterol, as well as other proteins involved in metabolism. AMPK and its role as an energy sensor has been reviewed in a variety of publications, including: Kemp et al (Trends Biochem. Sci. 1999 24:22-5), Hardie et al (Bioessays. 2001 23:1112-9), Musi et al (Curr. Drug Targets Immune Endocr. Metabol. Disord. 2002 2:119-27), Musi et al (Biochem. Soc. Trans. 2003 31:191-5) and Hardie (Endocrinology. 2003 144:5179-83) and Aschenbach (Sports Med. 2004 34:91-103), which publications are incorporated by reference.

The AMPK is a central regulator of energy homeostasis, which coordinates metabolic pathways and thus balances nutrient supply with energy demand. Because of the favorable physiological outcomes of AMPK activation on metabolism, AMPK has been considered to be an important therapeutic target for controlling human diseases including metabolic syndrome and cancer. As a cellular energy sensor, AMP-activated protein kinase (AMPK) is activated in response to a variety of conditions that deplete cellular energy levels, such as nutrient starvation (especially glucose), hypoxia and exposure to toxins that inhibit the mitochondrial respiratory chain complex. AMPK is a serine/threonine protein kinase complex consisting of a catalytic α-subunit (α1 and α2), a scaffolding β-subunit (β1 and β2) and a regulatory γ-subunit (γ1, γ2 and γ3).

The term “AMPK activation,” as used herein, refers to the phosphorylation state of AMPK or a direct target thereof. AMPK may be activated by modulation of a protein upstream of AMPK (e.g., the adponectin receptor, the leptin receptor, the a-adrenergic receptor, or the insulin receptor etc.) or by AMPK itself. AMPK activation may be determined by assaying AMPK itself or a downstream target of AMPK.

The term “AMP-activated protein kinase activator,” or “AMPK activator,” as used herein, refers to any compound or substance that activates AMP-activated protein kinase (AMPK). For example, EP application (EP Application No. 20205952.3) entitled “SUBSTITUTED RESORCYLIC ACID COMPOUNDS AS AMPK ACTIVATOR, COMPOSITIONS, METHODS AND USES THEREOF” describes compounds that bind directly to AMPK through the allosteric drug and metabolite (ADaM) site formed at the interface between the AMPK u and AMPK R subunits. These compounds are from the class of substituted resorcyclic acids and directly activate AMPK in cells.

An AMPK activator may be a direct AMPK activator or an indirect AMPK activator. In one embodiment, the AMPK activator is a direct AMPK activator. In one embodiment, the AMPK activator is an indirect AMPK activator.

In one embodiment, an AMPK activator may have potential as novel therapeutics for these diseases. In one embodiment, one type of AMPK activators is small molecules that mimic cellular AMP.

In one embodiment, the AMPK activator is cannabidiolic acid (CAS number 1244-58-2), CBDA, CBGA, CBNA or any of its derivatives. In one embodiment, the derivatives are any of the compounds with the general formula I.

In one embodiment, the AMPK activator is cannabidiol, CBDA, CBGA, CBNA or any of its derivatives. In one embodiment, the derivatives are any of the compounds with the general formula I.

The term “a sodium-glucose cotransporter-2 inhibitor,” or “SGLT2 inhibitor,” as used herein, refers to a compound or substance which exhibits an inhibitory effect on sodium-sugar carboxy-2 (SGLT2), in particular on human SGLT2. The inhibitory effect on hSGLT2 measured by IC50 is preferably 1,000 nM or less, more preferably 100 nM or less, and most preferably 50 nM or less. The IC50 value of an SGLT2 inhibitor is typically greater than 0.01 nM, or even greater than 0.1 nM. The inhibitory effect on hSGLT2 can be measured by methods known in the literature, in particular as described in application WO 2005/092877 or WO 2007/093610 (page 23/24); is hereby incorporated by reference. The term “SGLT2 inhibitor” also includes the pharmaceutically acceptable salts, hydrates and solvates thereof, including the respective crystal forms.

In one embodiment, the SGLT2 inhibitor is selected from the group consisting of dapagliflozin, canagliflozin, empagliflozin, artigliflozin, resmogliflozin, sergliflozin, phlorizin and their derivatives.

In one embodiment, the SGLT2 inhibitor may include a class of prescription medicines that are FDA-approved for use with diet and exercise to lower blood sugar in adults with type 2 diabetes. Medicines in the SGLT2 inhibitor class may include canagliflozin, dapagliflozin, and empagliflozin. They are available as single-ingredient products and also in combination with other diabetes medicines such as metformin. SGLT2 inhibitors lower blood sugar by causing the kidneys to remove sugar from the body through the urine.

Curr Opin Endocrinol Diabetes Obes. 2017 February; 24(1): 73-79. doi:10.1097/MED to Hsia et al provides a list of compounds as the SGLT2 inhibitor.

The term “active ingredient,” as used herein refer to any AMP-activated protein kinase (AMPK) activator (e.g., CBDA, CBGA or CBNA) and/or a SGLT2 inhibitor according to the present invention.

The term “nutritional composition,” or “nutritional supplement,” as used herein, refers to a nutritional product that provides nutrients to an individual that may otherwise not be consumed in sufficient quantities by the individual. For instance, a nutritional composition or nutritional supplement of the present invention may include vitamins, minerals, fiber, fatty acids, or amino acids. Nutritional compositions or nutritional supplements of the present invention may for example be provided in the form of a pill, a tablet, a lozenge, a chewy capsule or tablet, a tablet or capsule, or a powder supplement that can for example be dissolved in water or sprinkled on food.

In one embodiment, nutritional compositions or nutritional supplements of the present invention may provide selected nutrients while not representing a significant portion of the overall nutritional needs of a subject. Typically, they do not represent more than 0.1%, 1%, 5%, 10% or 20% of the daily energy need of a subject. A nutritional composition or nutritional supplement of the present invention may be used in any subject, such as a subject during pregnancy, e.g., as a maternal supplement.

As used herein, an “effective amount,” or “pharmaceutically effective amount,” as used herein, refers to an amount that prevents a deficiency, treats a disease or medical condition in an individual or, more generally, reduces symptoms, manages progression of the diseases or provides a nutritional, physiological, or medical benefit to the individual. The relative terms “promote,” “improve,” “increase,” “enhance” and the like refer to the effects of a composition comprising an AMP-activated protein kinase (AMPK) activator (e.g., CBDA, CBGA or CBNA) and a sodium-glucose cotransporter-2 (SGLT2) inhibitor disclosed herein relative to a composition lacking the AMP-activated protein kinase (AMPK) activator (e.g., CBDA, CBGA or CBNA) and/or the sodium-glucose cotransporter-2 (SGLT2) inhibitor, but otherwise identical.

The term “unit dosage form,” as used herein, refers to physically discrete units suitable as unitary dosages for human and animal subjects, each unit containing a predetermined quantity of the nutritional composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of powder in a sachet.

The term “nutritional product,” as used herein, refers to any product that can be used to provide nutrition to a subject. Typically, a nutritional product contains a protein source, a carbohydrate source and a lipid source.

The term “food product,” as used herein, refers to any kind of product that may be safely consumed by a human or an animal. A food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The food product may also contain anti-oxidants, stabilizers (when provided in solid form) or emulsifiers (when provided in liquid form).

The term “functional food product,” as used herein, refers to a food product providing an additional health-promoting or disease-preventing function to the individual.

The term “healthy ageing product,” as used herein, refers to a product providing an additional health-promoting or disease-preventing function related to healthy ageing to the individual.

The term “dairy products,” as used herein, refers to food products produced from milk or fractions of milk from animals such as cows, goats, sheep, yaks, horses, camels, and other mammals. Examples of dairy products are low fat milk (e.g., 0.1%, 0.5% or 1.5% fat), fat-free milk, milk powder, whole milk, whole milk products, butter, buttermilk, buttermilk products, skim milk, skim milk products, high milk-fat products, condensed milk, creme fraiche, cheese, ice cream and confectionery products, probiotic drinks or probiotic yoghurt type drinks.

The term “dairy alternative product,” as used herein, refers to products similar to dairy products but produced without milk.

The term “milk,” as used herein, is defined by Codex Alimentarius as the normal mammary secretion of milking animals obtained from one or more milkings without either addition to it or extraction from it, intended for consumption as liquid milk or for further processing.

The term “beverage product,” as used herein, refers to a nutritional product in liquid or semi-liquid form that may be safely consumed by an individual.

The term “diet product,” as used herein, refers to a food product with a restricted and/or reduced caloric content.

The term “pet food product,” as used herein, refers to a nutritional product that is intended for consumption by pets. A pet, or companion animal, as referenced herein, is to be understood as an animal selected from dogs, cats, birds, fish, rodents such as mice, rats.

As used herein, an “effective amount” is an amount that prevents a deficiency, treats a disease or medical condition in an individual or, more generally, reduces symptoms, manages progression of the diseases or provides a nutritional, physiological, or medical benefit to the individual.

All ingredients of the composition can be admixed together or alternatively the composition can be provided in the form of a kit of parts wherein ingredients or groups of ingredients are provided separately. These separate compositions may be intended to be consumed separately or together.

The descriptions of simple chains like methyl, pentyl, dodecyl are known to the person skilled in the art. For example, methyl is an alkyl derived from methane, containing one carbon atom bonded to three hydrogen atoms—CH₃. Pentyl is a five-carbon alkyl functional group (substituent) with chemical formula —C₅H₁₁. Dodecyl is a twelve-carbon alkyl functional group (substituent) with chemical formula —C₁₂H₂₅.

In all structures, the dot represents the point of insertion of the chain on the aromatic ring.

For example, if in Markush structure A, R2 is 3-methyl-2-buten-1-yl (1h), this corresponds to structure B:

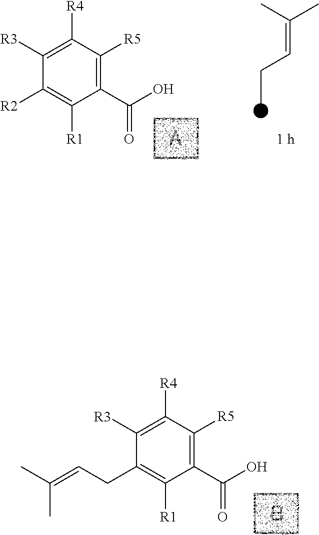

The term “C₅ isoprenoid,” as used herein, refers to the following substituents: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p).

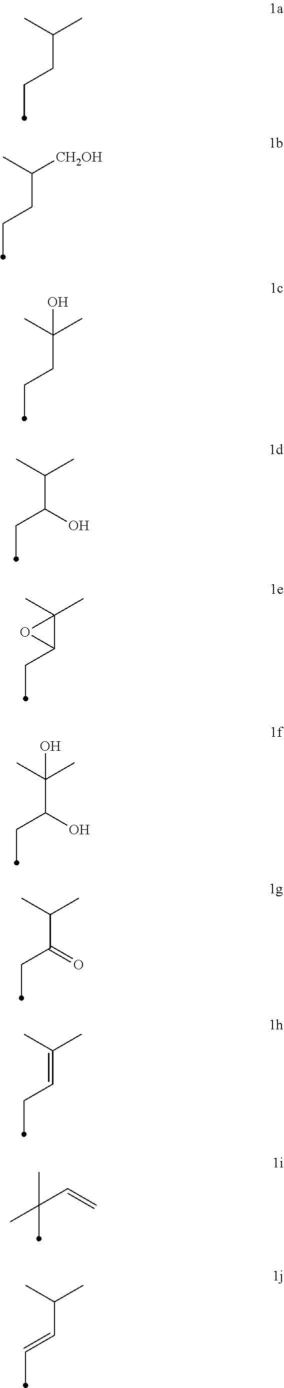

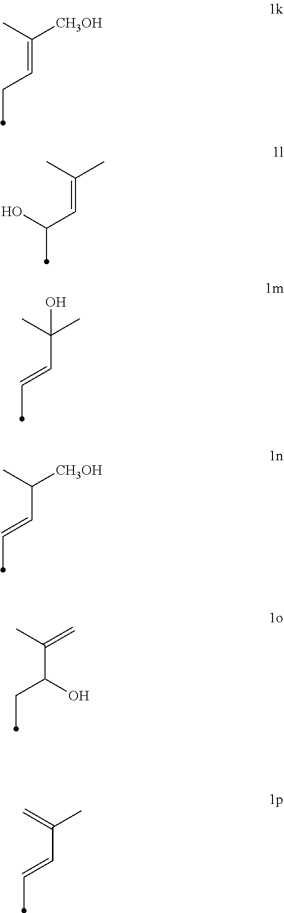

The term “C₁₀ isoprenoid,” as used herein, refers to the following substituents: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o).

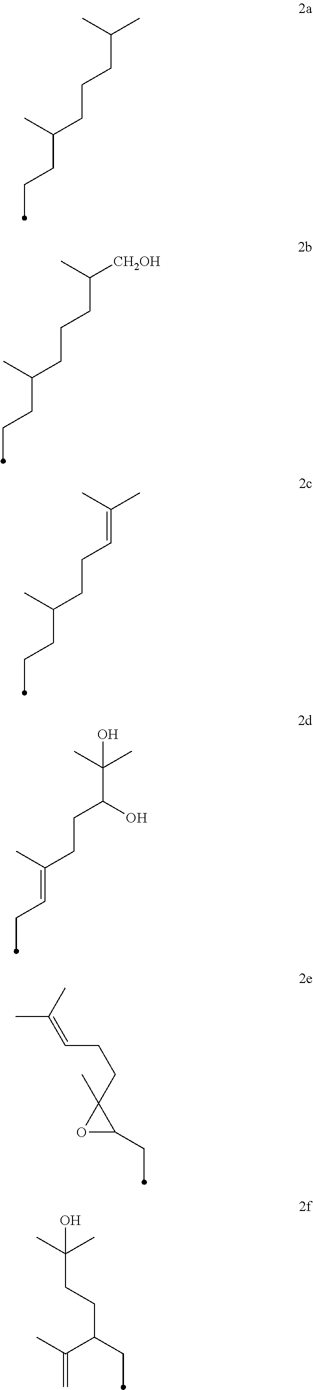

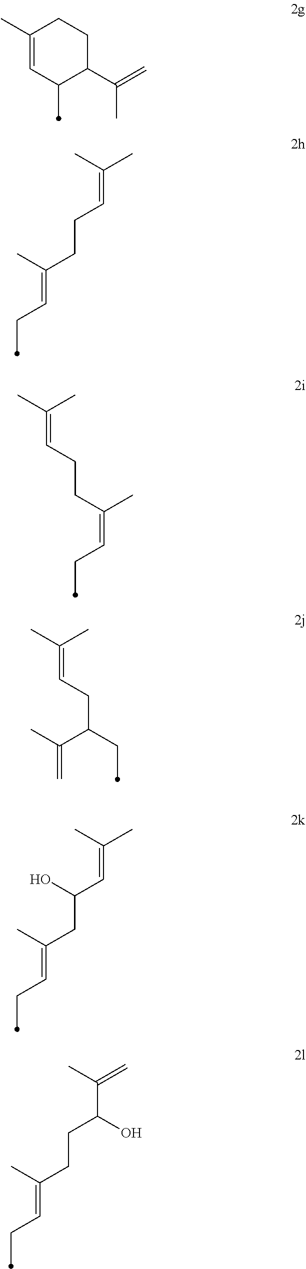

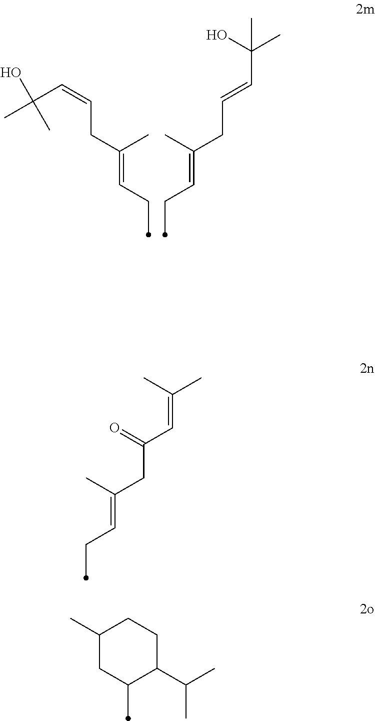

The term “C₁₅ isoprenoid,” as used herein, refers to the following substituents: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl) furan-3-yl]hex-2-en-1-yl (3d).

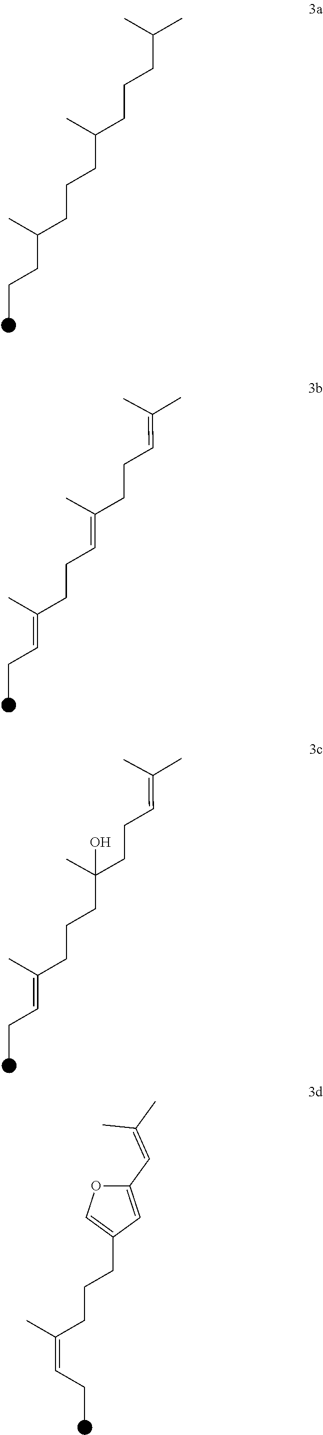

The term “C₂₀ isoprenoid,” as used herein, refers to the following substituents: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b).

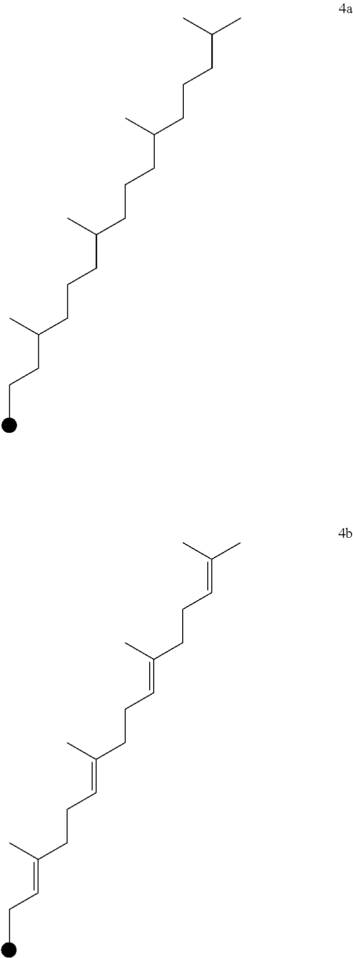

The term “alkyl,” as used herein, refers to a branched or unbranched saturated hydrocarbon chain having from 1 to 20 carbon atoms, or from 1 to 15 carbon atoms, or from 1 to 10 carbon atoms, or from 1 to 7 carbon atoms, or from 1 to 5 carbon atoms, or from 1 to 3 carbon atoms. The alkyl chain may be cyclic, in which case it would be known as “cycloalkyl” group.

Non-limiting examples of branched or unbranched alkyl chains include: methyl, ethyl, n-propyl, iso-propyl, n-butyl, iso-butyl, t-butyl, pentyl, hexyl, heptyl, nonyl, decyl, undecyl, tetradecyl, pentadecyl, heptadecyl, eicosyl.

A non-limiting example of cycloalkyl chain includes: 5-methyl-2-(1-methylethyl)cyclohexyl (2o)

The term “substituted alkyl,” refers to: 1) an alkyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙ R<a>, in which R<a> is alkyl, aryl or heteroaryl and n is 0, 1 or 2; One of the oxygen substituents of the alkyl chain can be joined by single bonds to two adjacent carbon atoms of the same alkyl chain, to form an epoxy group, i.e. a three-membered epoxide ring.

Non limiting examples of alkyl chains substituted by hydroxy, alkoxy, and/or acyloxy groups, or containing an epoxy group include: 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 8-hydroxy-3,7-dimethyloctyl (2b), hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p).

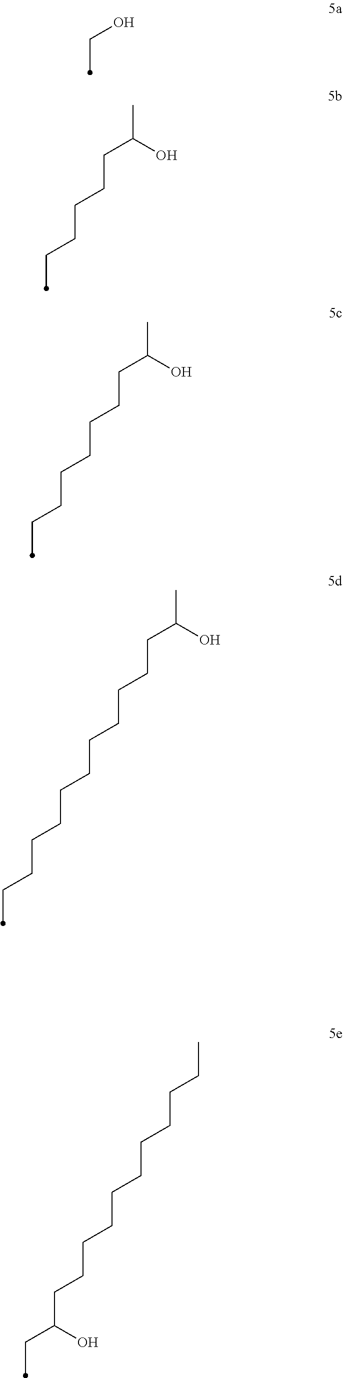

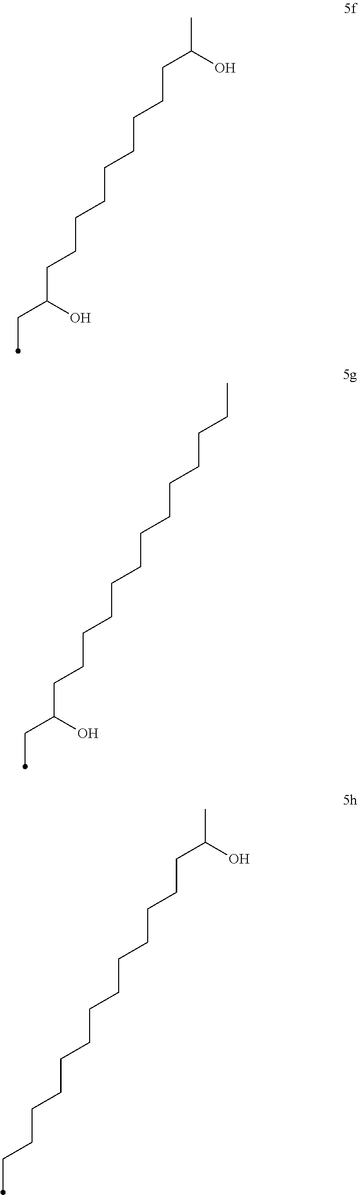

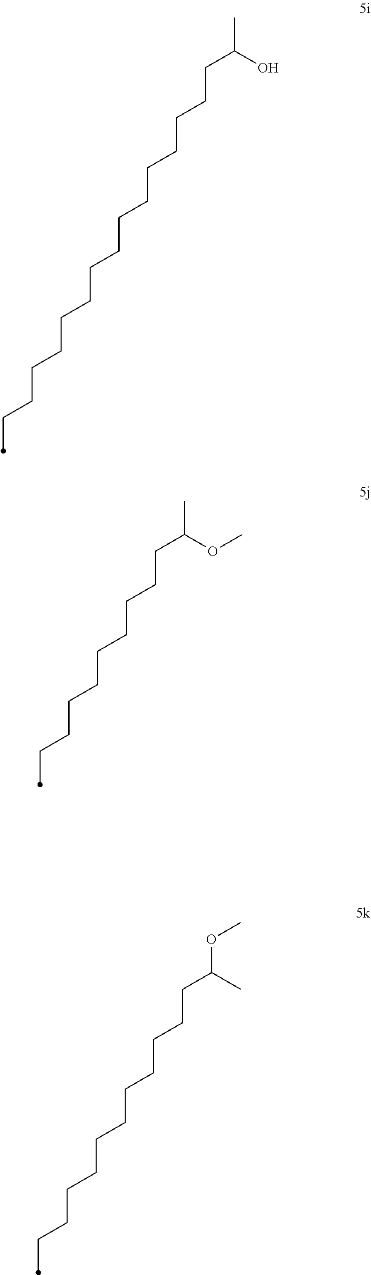

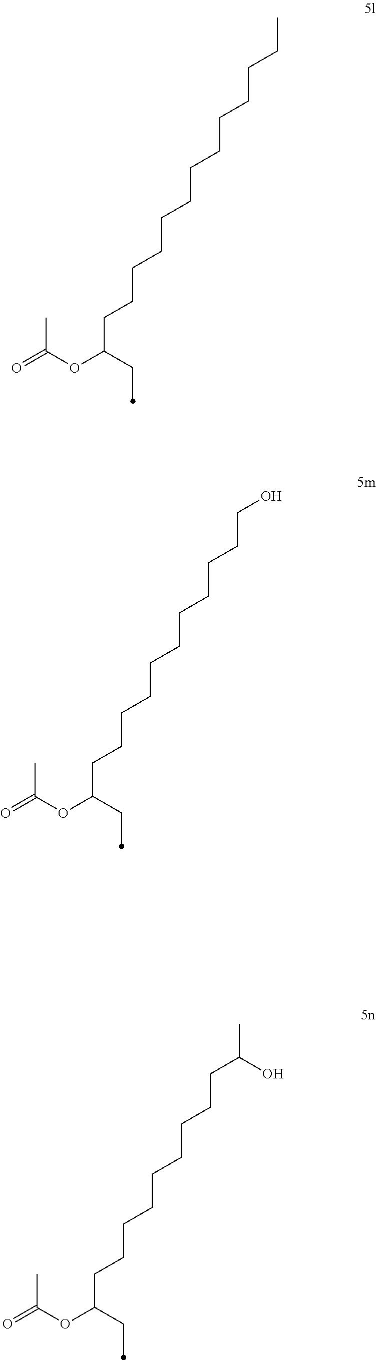

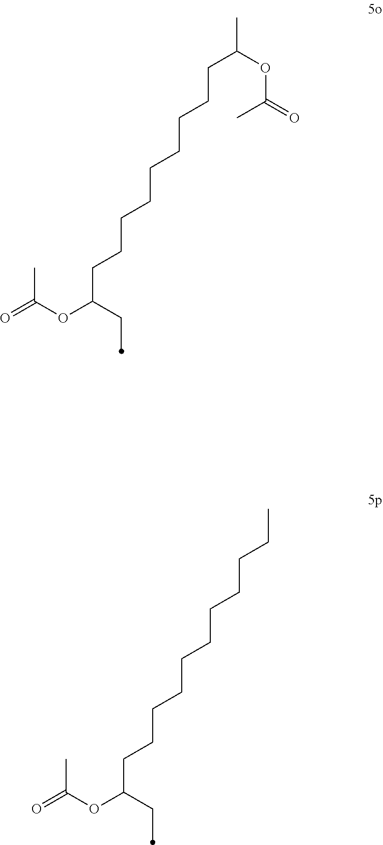

Non limiting examples of alkyl chains substituted by aryl/aryloxy groups include: Benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e).

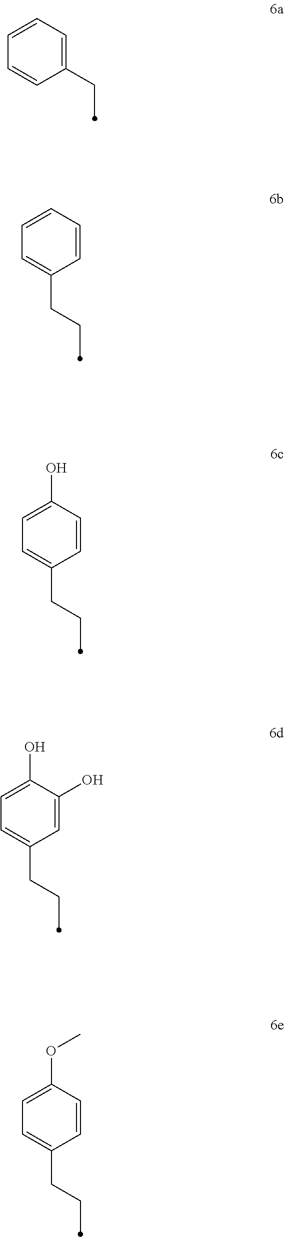

Non limiting examples of alkyl chains substituted by aryl/aryloxy and hydroxy groups include: 2-Hydroxy-2-phenylyethyl (7a), 2-hydroxy-2-(2-hydroxyphenyl)ethyl (7b).

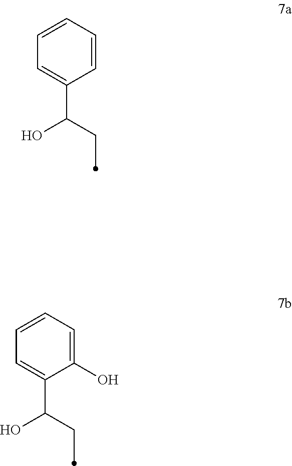

A non-limiting example of alkyl chain substituted by a heterocyclyl group includes: Tetrahydro-6-methyl-2H-pyran-2-yl)methyl (8a).

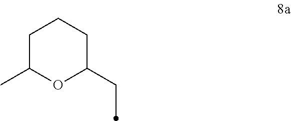

or
2) an alkyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) an alkyl chain as defined above that has both 1, 2, 3, 4 or 5 substituents as defined above and is also interrupted by 1-5 atoms (e.g. 1, 2, 3, 4 or 5 atoms) as defined above; or
4) an alkyl chain as defined above in which one or more of the methylene group is replaced by a carbonyl group to give an oxo group.

Non limiting examples of alkyl chain in which one or more of the methylene group is replaced by a carbonyl group include: 3-methyl-2-oxobutyl (1g), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 2-oxotridecyl (9g).

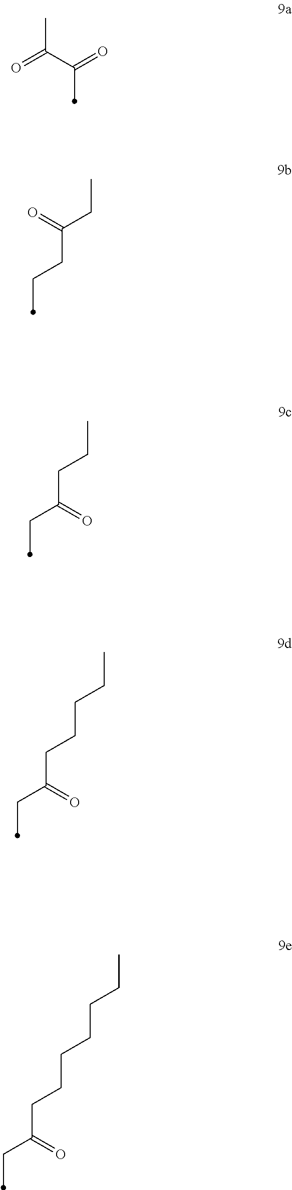

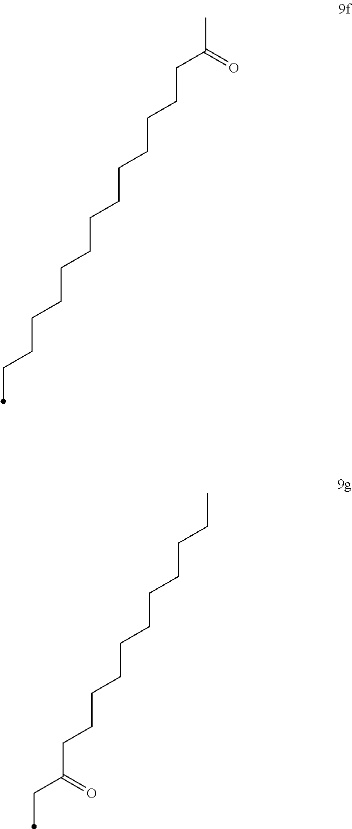

or
5) an alkyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non limiting examples of alkyl chain in which one of the methylene group is replaced by a carbonyl group to give an oxo group, and has a hydroxy substituent include: 1-hydroxy-2-oxopropyl (10a), 13-hydroxy-2-oxotridecyl (10b).

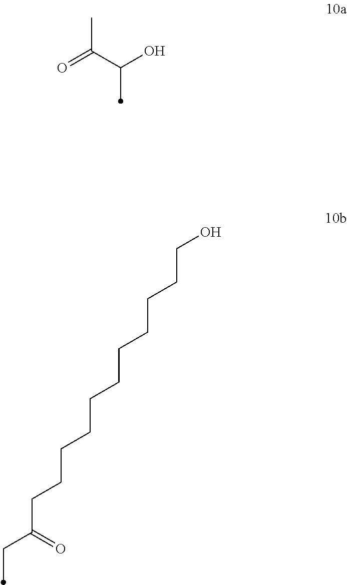

Non limiting examples of alkyl chains substituted by aryl/aryloxy, in which one of the methylene group is replaced by a carbonyl group to give an oxo group includes: 2-(4-hydroxyphenyl)-2-oxoethyl (11a), 2-oxo-2-phenylethyl (11b).

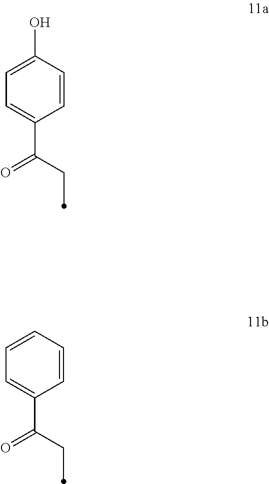

The term “alkenyl,” as used herein, refers to a type of alkyl chain as defined above, in which two atoms of the alkyl chain form a double bond that is not part of an aromatic group. That is, an alkenyl chain contains the pattern R—C(R)═C(R)—R, In one embodiment, R refers to the remaining portions of the alkenyl chain, which may be the same or different. The alkenyl moiety may be branched, straight chain, or cyclic (in which case, it would also be known as a “cycloalkenyl” group).

The term “substituted alkenyl” refers to:
1) an alkenyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)2-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
One of the oxygen substituents of the alkenyl chain can be joined by single bonds to two adjacent carbon atoms of the same alkenyl chain, to form an epoxy group, i.e. a three-membered epoxide ring.
or
2) an alkenyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) an alkenyl chain as defined above that has both 1, 2, 3, 4 or 5 substituents as defined above and is also interrupted by 1-5 atoms (e.g. 1, 2, 3, 4 or 5 atoms) as defined above;
or
4) an alkenyl chain as defined above in which one or more of the methylene group is replaced by a carbonyl group to give an oxo group.
or
5) an alkenyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non-limiting examples of alkenyl chains include: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 3,7-dimethyloct-6-en-1-yl (citronellyl) (2c), ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g).

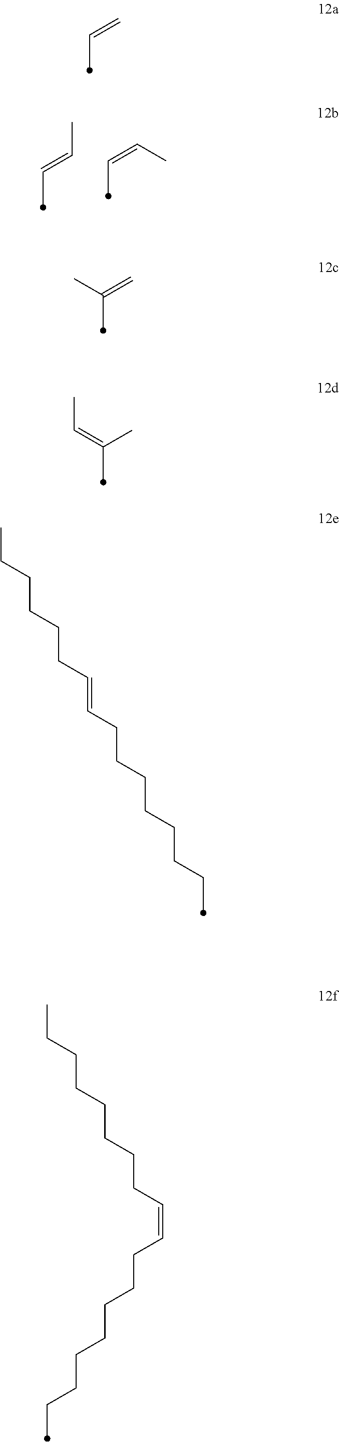

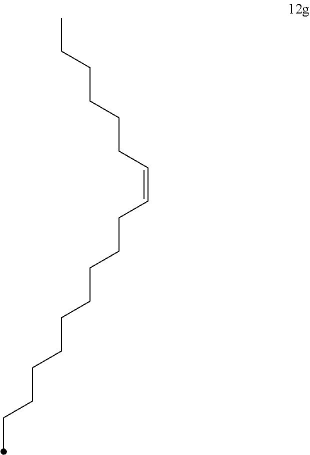

Non limiting examples of alkenyl chains substituted by hydroxy, and/or acyloxy groups, or containing an epoxy group include: 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 10-(acetyloxy)-8-pentadecenyl (13a).

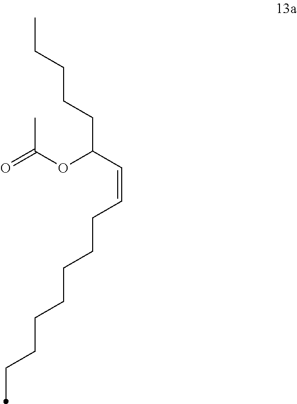

A non-limiting example of alkenyl chain substituted by an aryl group includes: 2-phenylethenyl (14a).

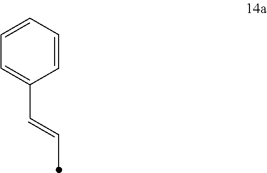

A non-limiting example of an alkenyl chain where one of the methylene is replaced by an oxo group includes: 1-hydroxymethylene-2-oxopropyl (15a).

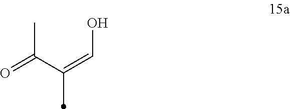

The term “alkynyl,” as used herein, refers to a type of alkyl chain as defined above in which two atoms of the alkyl chain form a triple bond. That is, an alkynyl chain contains the pattern R—C≡C—R, In one embodiment, R refers to the remaining portions of the alkynyl chain, which may be the same or different. Non-limiting examples of an alkynyl chain include —C≡CH, —C≡C—CH₃ and —C≡C—CH₂—CH₃. The “R” portion of the alkynyl moiety may be branched, straight chain, or cyclic. Alkynyl chains can be optionally substituted.

The term “substituted alkynyl” refers to:
1) an alkynyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
One of the oxygen substituents of the alkynyl chain can be joined by single bonds to two adjacent carbon atoms of the same alkynyl chain, to form an epoxy group, i.e. a three-membered epoxide ring.
or
2) an alkynyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) an alkynyl chain as defined above that has both 1, 2, 3, 4 or 5 substituents as defined above and is also interrupted by 1-5 atoms (e.g. 1, 2, 3, 4 or 5 atoms) as defined above;
or
4) an alkynyl chain as defined above in which one or more of the methylene group is replaced by a carbonyl group to give an oxo group.
or
5) an alkynyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

The term “polyalkenyl,” as used herein, refers to a chain in which more than one pair of atoms of the alkyl chain form a double bond that is not part of an aromatic group. That is, a polyalkenyl chain contains from 2 to 8 R—C(R)═C(R)—R patterns, In one embodiment, R refers to the remaining portions of the alkenyl chain, which may be the same or different. The polyalkenyl moiety may be branched, or straight chain.

Non-limiting examples of polyalkenyl chains include: 3-methyl-1,3-butadienyl (1p), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), (2E,6E)-3,7,11-trimethyl-2,6,10-dodecatrien-1-yl (farnesyl) (3b), (2E,6E,10E)-3,7,11,15-tetramethylhexadeca-2,6,10,14-tetraen-1-yl (geranylgeranyl) (4b), 8,11-heptadecadien-1-yl (16a).

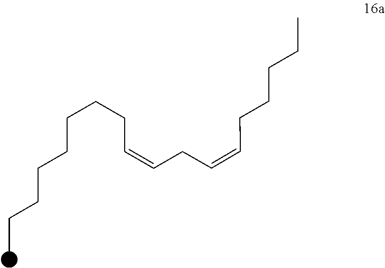 [SMILES: CCCCC/C=C\C/C=C\CCCCCCCC]

The polyalkenyl moiety containing two double bonds may be cyclic (in which case, it would also be known as a “cyclodialkenyl” group). Non limiting example of cyclodialkenyl groups include cyclopentadiene and cyclohexadiene groups. Polyalkenyl chains can be optionally substituted.

The term “substituted polyalkenyl,” as used herein, refers to:
1) a polyalkenyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙ R<a>, in which R<a> is alkyl, aryl or heteroaryl and n is 0, 1 or 2;
One of the oxygen substituent of the polyalkenyl chain can be joined by single bonds to two adjacent carbon atoms of the same polyalkenyl chain, to form an epoxy group, i.e. a three-membered epoxide ring. One of the oxygen substituent of the polyalkenyl chain can from a five-membered aromatic ring with four carbon atoms of the same chain, resulting in. a furan ring.
or
2) a polyalkenyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙ R<a>, in which R<a> is alkyl, aryl or heteroaryl and n is 0, 1 or 2;
or
3) a polyalkenyl chain as defined above that has both 1, 2, 3, 4 or 5 substituents as defined above and is also interrupted by 1-5 atoms (e.g. 1, 2, 3, 4 or 5 atoms) as defined above;
or
4) a polyalkenyl chain as defined above in which one or more of the methylene group is replaced by a carbonyl group to give an oxo group.
or
5) a polyalkenyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

Non limiting examples of polyalkenyl chains substituted by a hydroxy group, or containing a furan ring include: 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d).

Non limiting examples of linear or cyclized polyalkenyl chains in which one of the methylene group is replaced by a carbonyl group to give an oxo group, include: 3,7-dimethyl-5-oxo-2,6-octadienyl (2n), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c).

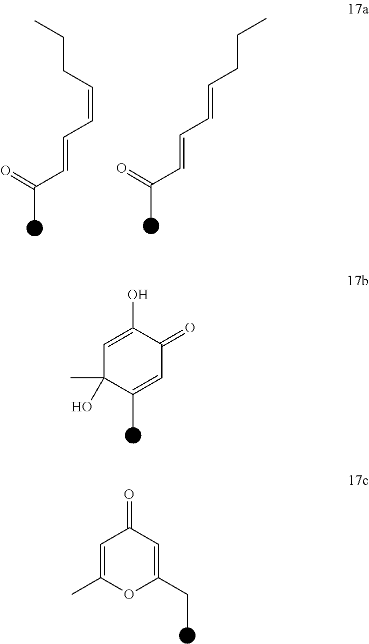

A non limiting example of a polyalkenyl chain substituted by an aryloxy group includes 8-(3,4-dihydroxyphenyl)-4,7-octadien-1-yl (18a).

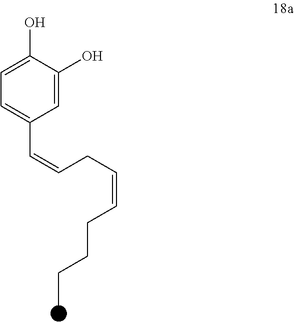 [SMILES: CCCC/C=C\C/C=C\c1ccc(O)c(O)c1]

The term polyalkynyl refers to a chain in which more than one pair of atoms of the alkyl chain form a triple bond. That is, a polyalkynyl chain contains from 2 to 8 R—C≡C—R patterns, In one embodiment, R refers to the remaining portions of the alkynyl chain, which may be the same or different. Non-limiting example of a polyalkynyl chain include —CH₂—CH₂—C≡C—C≡CH. The “R” portion of the polyalkynyl moiety may be branched, straight chain, or cyclic.

The term “substituted polyalkynyl,” as used herein, refers to:
1) a polyalkynyl chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
2) a polyalkynyl chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙ R<a>, in which R<a> is alkyl, aryl or heteroaryl and n is 0, 1 or 2;
or
3) a polyalkynyl chain as defined above that has both 1, 2, 3, 4 or 5 substituents as defined above and is also interrupted by 1-5 atoms (e.g. 1, 2, 3, 4 or 5 atoms) as defined above;
or
4) a polyalkynyl chain as defined above in which one or more of the methylene group is replaced by a carbonyl group to give an oxo group.
or
5) a polyalkynyl chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

The term “polyunsaturated,” as used herein, refers to a chain in which at least one pair of atoms of the alkyl chain form a double bond and one pair of atoms of the alkyl chain form a triple bond. That is, a polyunsaturated chain contains both R—C(R)═C(R)—R and R—C≡C—R patterns, In one embodiment, R refers to the remaining portions of the polyunsaturated chain, which may be the same or different and the total number of unsaturated bonds may vary from 2 to 8. Non-limiting examples this type of polyunsaturated chain include —CH₂—CH═CH—C≡CH. The “R” portion of the polyunsaturated moiety may be branched, straight chain, or cyclic.

The term “substituted polyunsaturated,” as used herein, refers to:
1) a polyunsaturated chain as defined above, having 1, 2, 3, 4 or 5 substituents, (in some embodiments, 1, 2 or 3 substituents) selected from the group consisting of alkyl; alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙ R<a>, in which R<a> is alkyl, aryl or heteroaryl and n is 0, 1 or 2;
or
2) a polyunsaturated chain as defined above that is interrupted by 1-5 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)n R<a>, in which R<a> is alkyl, aryl or heteroaryl and n is 0, 1 or 2;
or
3) a polyunsaturated chain as defined above that has both 1, 2, 3, 4 or 5 substituents as defined above and is also interrupted by 1-5 atoms (e.g. 1, 2, 3, 4 or 5 atoms) as defined above;
or
4) a polyunsaturated chain as defined above in which one or more of the methylene group is replaced by a carbonyl group to give an oxo group.
or
5) a polyunsaturated chain as defined above in which one of the methylene group is replaced by a carbonyl group to give an oxo group, and has 1, 2, 3, 4 or 5 substituents as defined above, or is interrupted by 1-5 atoms (e.g. 1, 2, 3, 4 or 5 atoms) as defined above or has both 1, 2, 3, 4 or 5 substituents as defined above and is also interrupted by 1-5 atoms (e.g. 1, 2, 3, 4 or 5 atoms) as defined above.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be optionally substituted. Rings can form part of a ring system. As used herein, the term “ring system” refers to two or more rings, In one embodiment, two or more of the rings are fused. The term “fused,” as used herein, refers to structures in which two or more rings share one or more bonds.

The term “halogen,” as used herein, refers to a fluorine atom, a chlorine atom, a bromine atom or an iodine atom.

The term “glycoside,” as used herein, refers to a compound in which at least one sugar is bound to another functional group via a glycosidic bond. Typically the glycosidic chain can comprise 1 to 4 sugar units.

The term “glycosidic bond,” as used herein, refers to a bond formed between the hemiacetal or hemiketal group of a sugar and the chemical group of a compound. The chemical group can be —OH (O-glycoside), or —CR1R2R3 (C-glycoside).

The terms “acylated O-glycoside” and “acylated C-glycoside” refer to a compound in which at least one hydroxyl of the glycosidic chain is esterified by an organic acid. Typical examples or organic acid may comprise acetic, substituted benzoic, cinnamic (caffeic, ferulic, p-coumaric), and/or phenylpropanoic (dihydrocaffeic) acids.

The terms “sulfated O-glycoside” and “sulfated C-glycoside” refer to a compound in which at least one hydroxyl of the glycosidic chain is esterified by sulfuric acid.

The term “methylene dioxy,” as used herein, refers to functional group with the structural formula R—O—CH₂—O—R′, connected to the rest of a molecule by two chemical bonds.

The term “analogue,” as used herein, refers to a compound having a structure similar to that of another one, but differing from it in respect of a certain component. The term “derivative,” as used herein, refers to any compound that can be imagined to arise or is actually be synthesized from a parent compound by replacement of one or more atoms with another atom or group of atoms.

It is understood that according to certain embodiments, the compound of the invention or composition thereof may be a nutraceutical composition, pharmaceutical composition, functional food, functional nutrition product, medical food, medical nutrition product, or a dietary supplement.

The terms “nutraceutical” combines the words “nutrition” and “pharmaceutical”. It is a food or food product that provides health and medical benefits, including the prevention and treatment of a condition, disorder, or disease. A nutraceutical is a product isolated or purified from foods that is generally sold in medicinal forms not usually associated with food. A nutraceutical is demonstrated to have a physiological benefit or provide protection against a condition, disorder, or disease. Such products may range from isolated nutrients, dietary supplements and specific diets to genetically engineered foods, herbal products, and processed foods such as cereals, soups, and beverages.

The term “nutraceutical,” as used herein, denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

Moreover, a multi-vitamin and mineral supplement may be added to nutraceutical compositions of the invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions of the invention may be in any galenic form that is suitable for administering to the body, especially in any form that is conventional for oral administration, e.g. in solid forms such as food or feed, food or feed premix, fortified food or feed, tablets, pills, granules, dragees, capsules and effervescent formulations such as powders and tablets, or in liquid forms, such as solutions, emulsions or suspensions as e.g. beverages, pastes and oily suspensions. The pastes may be incorporated in hard or soft shell capsules, whereby the capsules feature e.g. a matrix of (fish, swine, poultry, cow) gelatine, plant proteins or lignin sulfonate. Examples for other application forms are those for transdermal, parenteral or injectable administration. The dietary and pharmaceutical compositions may be in the form of controlled (delayed) release formulations.

Beverages encompass non-alcoholic and alcoholic drinks as well as liquid preparations to be added to drinking water and liquid food. Non-alcoholic drinks are e.g. soft drinks, sports drinks, fruit juices, teas and milk-based drinks. Liquid foods are e.g. soups and dairy products. The nutraceutical composition comprising the compound of the invention may be added to a soft drink, an energy bar, or a candy.

If the nutraceutical composition is a pharmaceutical formulation and the composition further contains pharmaceutically acceptable excipients, diluents or adjuvants then standard techniques may be used for their formulation, as e.g., disclosed in Remington's Pharmaceutical Sciences, 20th edition Williams & Wilkins, PA, USA. For oral administration, tablets and capsules are preferably used which contain a suitable binding agent, e.g., gelatine or polyvinyl pyrrolidone, a suitable filler, e.g. lactose or starch, a suitable lubricant, e.g. magnesium stearate, and optionally further additives.

The term “functional food,” “functional nutrition product,” “medical food,” or “medical nutrition product,” as used herein, refers to any healthy food claimed to have a health-promoting or disease-preventing property beyond the basic function of supplying nutrients. The general category of functional foods includes processed food or foods fortified with health-promoting additives, like “vitamin-enriched” products.

The terms “food,” “food product” and “food composition” or “diet product” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

A dietary supplement, also known as food supplement or nutritional supplement, is a preparation intended to supplement the diet and provide nutrients, such as vitamins, minerals, fibre, fatty acids, or amino acids that may be missing or may not be consumed in sufficient quantities in a person's diet. Some countries define dietary supplements as foods, while in others they are defined as drugs or natural health products. Supplements containing vitamins or dietary minerals are included as a category of food in the Codex Alimentarius, a collection of internationally recognized standards, codes of practice, guidelines and other recommendations relating to foods, food production and food safety. These texts are drawn up by the Codex Alimentarius Commission, an organization that is sponsored by the Food and Agriculture Organization of the United Nations (FAO) and the World Health Organization (WHO).

Compositions intended for an animal, include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

“Pet food” or “pet treat compositions” comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof. The compositions may further comprise from about 5% to about 40% fat. The compositions may further comprise a source of carbohydrate. The compositions may comprise from about 15% to about 60% carbohydrate. Examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

As used herein, the term “diabetes” includes insulin-dependent diabetes mellitus (i.e. IDDM, also known as type 1 diabetes) non-insulin-dependent diabetes mellitus (i.e. NIDDM, also known as type 2 diabetes), and prediabetes. Type 1 diabetes is the result of an absolute deficiency of insulin, the hormone which regulates glucose utilization. Type 2 diabetes often occurs in the face of normal, or even elevated levels of insulin and appears to be the result of the inability of tissues to respond appropriately to insulin. This is termed “insulin resistance”. Most type 2 diabetic patients are also overweight or obese. One of the criteria for diagnosing diabetes is the fasting plasma glucose level. A diabetic subject has a fasting plasma glucose level of greater than or equal to 126 mg/dl. A prediabetic subject is someone suffering from prediabetes. A prediabetic subject is a subject with impaired fasting glucose (a fasting plasma glucose level of greater than or equal to 100 mg/dl and less than 126 mg/dl); or impaired glucose tolerance (a 2-hour plasma glucose level of >140 mg/dl and <200 mg/dl); or insulin resistance, resulting in an increased risk of developing diabetes. Prevention of type 2 diabetes includes treatment of prediabetes.

As used herein, the term “dyslipidemia” encompasses abnormal levels of any lipid fractions as well as specific lipoprotein abnormalities. For example, it refers to elevation of plasma cholesterol and/or elevation of triglycerides and/or elevation of free fatty acids and/or low high-density lipoprotein (HDL) level and/or high low-density lipoprotein (LDL) level and/or high very low-density lipoprotein (VLDL) level. Dyslipidemia may for example contribute to the development of atherosclerosis and ultimately symptomatic vascular disease including coronary heart disease. Dyslipidemia may or may not be associated with diabetes.

As used herein, the term “metabolic disorder” encompasses any abnormal chemical and enzymatic reactions disrupting normal metabolism due to environmental and genetic factors (environmental factors include physical activity, nutrition), leading to excessive levels or deficiency of certain substances and dysfunction of energy homeostasis. Non-limiting examples of metabolic disorders include diabetes, dyslipidemia, hypertension, being overweight, obesity, and any combination thereof.

As used herein, the term “AMPK-related diseases” includes pathologic or pathogenomic conditions in which the activation of AMPK provides a salutary effect. Examples of such diseases or conditions include obesity, diabetes, metabolic syndrome, acute inflammatory lung injury, heart disease, reperfusion ischemia, cancer, aging, retinal degeneration, cardiac hypertrophy, non-alcoholic fatty liver disease, hypertension, albuminuria, sporadic Alzheimer's disease, muscular dystrophy, and osteoarthritis. In addition, the term “AMPK-related conditions” includes conditions where the activation of AMPK improves the condition associated with the primary “AMPK-related disease”. For example, diabetic nephropathy (Salotto et al. (2017) J. Pharma and Expt Thera. 361:303-311) or diabetic neuropathy are “AMPK-related conditions” which may be associated with the “AMPK-related disease” of diabetes.

The term “prevention” or “preventing,” as used herein, refers to reduction of risk and/or severity of a condition, disorder, or disease.

The term “treatment,” “treating,” “treat,” “attenuate,” or “alleviate,” as used herein, refers to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in a subject not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers, (see, e.g., Nishina, et al., Metab. 43:554-558, 1994; Grundy and Barnett, Dis. Mon. 36:641-731, 1990; Rissanen, et al., British Medical Journal, 301:835-837, 1990).

The term “obesity related disorders,” as used herein, refers to those diseases or conditions where excessive body weight or high “body mass index (BMI)” has been implicated in the progression or suppression of the disease or condition. Representative examples of obesity related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 13th Ed., McGraw Hill Companies Inc., New York (1994). Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

The term “subject,” “individual,” or “patient,” as used herein, refers to any animal, including a human, that could benefit from one or more of the compounds, compositions or methods disclosed herein. Generally, the subject is a human or an avian, bovine, canine, equine, feline, hircine, lupine, murine, ovine or porcine animal. The “companion animal,” as used herein, refers to any domesticated animal, and includes, without limitation, cats, dogs, rabbits, guinea pigs, ferrets, hamsters, mice, gerbils, horses, cows, goats, sheep, donkeys, pigs, and the like. Preferably, the subject is a human or a companion animal such as a dog or cat. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly subjects. For other animals, an “older adult” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An elderly cat or dog has an age from birth of at least about 7 years.

In one embodiment, the term “subject,” as used herein, refers to a mammal. Mammal includes, but is not limited to, rodents, aquatic mammals, domestic animals such as dogs and cats, farm animals such as sheep, pigs, cows and horses, and humans. In one embodiment, the mammal may be a cat, a dog or a human. The human may be a woman, for example, a woman who is trying to get pregnant, or who is pregnant. In one embodiment of the invention, the subject is a mammal selected from the group consisting of a cat, a dog and, a human. For example, the subject may be an old human.

The term “empagliflozin,” as used herein, refers to is an antidiabetic medication (sold under the brand name Jardiance among others) used to improve glucose control in people with type 2 diabetes, used to reduce the risk of cardiovascular death in adults with type 2 diabetes and established cardiovascular disease, used to reduce the risk of death and hospitalization in people with heart failure and low ejection fraction, and used to reduce the risk of cardiovascular death and hospitalization for heart failure in adults. It can be prescribed instead of metformin and has benefits over sulfonylureas. It may be used together with other medications such as metformin or insulin. It may not be recommended for type 1 diabetes. Empagliflozin is an inhibitor of the sodium glucose co-transporter-2 (SGLT-2), and works by increasing sugar lost in the urine. The IUPAC name for empagliflozin is (2S,3R,4R,5S,6R)-2-[4-Chloro-3-[[4-[(3S)-oxolan-3-yl]oxyphenyl]methyl]phenyl]-6-(hydroxymethyl)oxane-3,4,5-triol.

The term “canagliflozin,” as used herein, refers to a medication (sold under the brand name Invokana among others) used to treat type 2 diabetes. It is a third-line medication to be tried after metformin, a first-line medication for type 2 diabetes. It is used together with exercise and diet. It is not recommended in type 1 diabetes. Canagliflozin is a sodium-glucose cotransporter-2 (SGLT2) inhibitor. It works by increasing the amount of glucose lost in the urine. The IUPAC name for canagliflozin is (2S,3R,4R,5S,6R)-2-{3-[5-[4-Fluoro-phenyl)-thiophen-2-ylmethyl]-4-methyl-phenyl}-6-hydroxymethyl-tetrahydro-pyran-3,4,5-triol.

The term “dapagliflozin,” or “Dapa,” as used herein, refers to is a medication (sold under the brand names Farxiga (US) and Forxiga (EU) among others) used to treat type 2 diabetes. It is also used to treat adults with certain kinds of heart failure and chronic kidney disease. Dapagliflozin is a sodium-glucose co-transporter-2 (SGLT-2) inhibitor and works by removing sugar from the body with the urine. The IUPAC name for dapagliflozin is (2S,3R,4R,5S,6R)-2-[4-Chloro-3-(4-ethoxybenzyl)phenyl]-6-(hydroxymethyl)tetrahydro-2H-pyran-3,4,5-triol.

The term “ertugliflozin,” as used herein, refers to a medication (sold under the brand name Steglatro) for the treatment of type 2 diabetes. Ertugliflozin is a sodium/glucose cotransporter 2 (SGLT2) inhibitor and is in the class of drugs known as gliflozins. The IUPAC name for ertugliflozin is (1S,2S,3S,4R,5S)-5-[4-Chloro-3-(4-ethoxybenzyl)phenyl]-1-(hydroxymethyl)-6,8-dioxabicyclo[3.2.1]octane-2,3,4-triol.

The term “phlorizin,” as used herein, refers to a glucoside of phloretin, a dihydrochalcone. A white solid, samples often appear yellow owing to impurities. It is of sweet taste and contains four molecules of water in the crystal. Above 200° C., it decomposes to give rufin. It is poorly soluble in ether and cold water, but soluble in ethanol and hot water. Upon prolonged exposure to aqueous solutions phlorizin hydrolyzes to phloretin and glucose. The IUPAC name of phlorizin is 1-(2,4-Dihydroxy-6-{[(2S,3R,4S,5S,6R)-3,4,5-trihydroxy-6-(hydroxymethyl)oxan-2-yl]oxy}phenyl)-3-(4-hydroxyphenyl)propan-1-one. It is also called Isosalipurposide.

### Embodiments

### Compositions

An aspect of the present disclosure is a composition comprising a combination of an AMP-activated protein kinase (AMPK) activator (e.g., CBDA, CBGA, CBNA or derivatives or analogues thereof) and a sodium-glucose cotransporter-2 (SGLT2) inhibitor for synergistically activating AMPK and improving metabolic health (e.g., treating or preventing metabolic disorder or obesity related disorders).

Applicant surprisingly found that administering a subject in need thereof a composition comprising a combination of an AMP-activated protein kinase (AMPK) activator (e.g., CBDA, CBGA, CBNA or derivatives or analogues thereof) and a sodium-glucose cotransporter-2 (SGLT2) inhibitor led to improved performance of activated AMPK as compared to administering an AMP-activated protein kinase (AMPK) activator (e.g., CBDA, CBGA, CBNA or derivatives or analogues thereof) or a sodium-glucose cotransporter-2 (SGLT2) inhibitor alone, thus synergistically activating AMPK for improving metabolic health. For example, Applicant surprisingly found that administering the subject a composition comprising a combination of an AMP-activated protein kinase (AMPK) activator (e.g., CBDA, CBGA, CBNA or derivatives or analogues thereof) and a sodium-glucose cotransporter-2 (SGLT2) inhibitor can lead to at least 1% overall improvement on glucose clearance in the subject as compared to administering an AMP-activated protein kinase (AMPK) activator (e.g., CBDA, CBGA, CBNA or derivatives or analogues thereof) or a sodium-glucose cotransporter-2 (SGLT2) inhibitor alone. Further, Applicant surprisingly found that administering the subject a composition comprising a combination of an AMP-activated protein kinase (AMPK) activator (e.g., CBDA) and a sodium-glucose cotransporter-2 (SGLT2) inhibitor can lead to at least 1% overall improvement on glucose clearance in the subject as compared to the total effect on glucose clearance of an AMP-activated protein kinase (AMPK) activator (e.g., CBDA, CBGA, CBNA or derivatives or analogues thereof) and a sodium-glucose cotransporter-2 (SGLT2) inhibitor.

In an aspect, the present disclosure relates to a composition for use in the activation of AMPK in a subject comprising a first compound and a second compound. The subject is a mammal such as a cat, a dog or a human. In one embodiment, the subject is a female human or a male human. In one preferred embodiment, the subject is an old female human or an old male human.

In one embodiment, the first compound is an AMP-activated protein kinase (AMPK) activator or its derivatives. In one embodiment, the second compound is a sodium-glucose cotransporter-2 (SGLT2) inhibitor or its derivatives.

In one embodiment, the first compound is a direct AMP-activated protein kinase (AMPK) activator or its derivatives. In one embodiment, the second compound is a sodium-glucose cotransporter-2 (SGLT2) inhibitor selected from the group consisting of dapagliflozin, canagliflozin, empagliflozin, artigliflozin, resmogliflozin, sergliflozin, phlorizin and their derivatives.

In one embodiment, the first compound is a substituted resorcyclic acid as a direct AMP-activated protein kinase (AMPK) activator or its derivatives. In one embodiment, the second compound is a sodium-glucose cotransporter-2 (SGLT2) inhibitor selected from the group consisting of dapagliflozin, canagliflozin, empagliflozin, artigliflozin, resmogliflozin, sergliflozin, phlorizin and their derivatives.

In one embodiment, the composition for use in the activation of AMPK, the composition comprising a first compound; and a second compound; wherein the first compound has the general formula I,

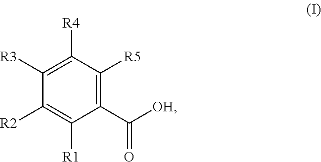

wherein R1 and R3 are each independently OH; OCH₃; O-aliphatic saturated or unsaturated acyl, O-glycoside; acylated O-glycoside; sulfated O-glycoside; or a sulfate;
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl; optionally a OCH₃ group can cyclize with a neighboring OH group to form a methylene dioxy bridge; or a derivative or analogue thereof;
wherein the second compound is a sodium-glucose cotransporter-2 (SGLT2) inhibitor.

In one embodiment, the sodium-glucose cotransporter-2 (SGLT2) inhibitor is selected from the group consisting of empagliflozin, canagliflozin, dapagliflozin, and ertugliflozin.

In one embodiment, the sodium-glucose cotransporter-2 (SGLT2) inhibitor is selected from the group consisting of empagliflozin, canagliflozin, phlorizin and dapagliflozin.

In one embodiment, the sodium-glucose cotransporter-2 (SGLT2) inhibitor comprises dapagliflozin.

In one embodiment, the sodium-glucose cotransporter-2 (SGLT2) inhibitor is dapagliflozin.

In one embodiment, the sodium-glucose cotransporter-2 (SGLT2) inhibitor is phlorizin.

In one embodiment, the first compound has the general formula I,

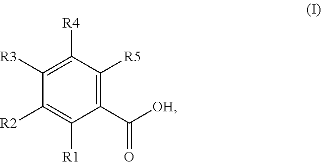

wherein:
R1 and R3 are each independently OH; OCH₃; O-aliphatic saturated or unsaturated acyl, O-glycoside; acylated O-glycoside; or sulfated O-glycoside;
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; or a sulfonate; an optionally substituted and/or optionally branched C₁ to C₂₀ alkyl; an optionally substituted and/or optionally branched, C₂ to C₂₀ alkenyl; an optionally substituted and/or optionally branched, C₄ to C₁₀ polyalkenyl; an optionally substituted and/or optionally branched C₂ to C₂₀ alkynyl, or an optionally substituted and/or optionally branched C₄ to C₂₀ polyalkynyl;
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; or a sulfonate; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; or a sulfonate; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl; optionally a OCH₃ group can cyclize with a neighboring OH group to form a methylene dioxy bridge; or a derivative or analogue thereof.

In one embodiment, the first compound has the general formula I,

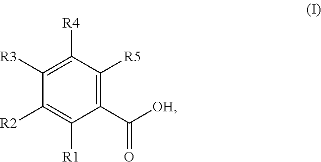

wherein:
R1 and R3 are each independently OH; OCH₃; O-aliphatic saturated or unsaturated acyl, O-glycoside; or acylated O-glycoside;
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; or a nitro; an optionally substituted and/or optionally branched C₁ to C₂₀ alkyl; an optionally substituted and/or optionally branched, C₁₀ to C₂₀ alkenyl; an optionally substituted and/or optionally branched, C₁₀ to C₂₀ polyalkenyl; an optionally substituted and/or optionally branched C₂ to C₂₀ alkynyl, or an optionally substituted and/or optionally branched C₄ to C₂₀ polyalkynyl;
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; or a nitro; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; or a nitro; an optionally branched C₁ to C₂₀ alkyl; an optionally branched, C₂ to C₂₀ alkenyl; an optionally branched, C₄ to C₂₀ polyalkenyl; an optionally branched C₂ to C₂₀ alkynyl, or an optionally branched C₄ to C₂₀ polyalkynyl; optionally a OCH₃ group can cyclize with a neighboring OH group to form a methylene dioxy bridge; or a derivative or analogue thereof.

In another embodiment, the first compound has the general formula I,

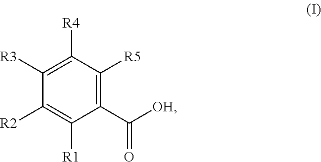

wherein:
R1 and R3 are each independently OH; OCH₃; O-aliphatic saturated or unsaturated acyl, O-glycoside; or acylated O-glycoside;
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; or an ester; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; or an ester; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl;
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; or an ester; an optionally substituted and/or optionally branched C₁ to C₂₀ alkyl; an optionally substituted and/or optionally branched, C₂ to C₂₀ alkenyl; an optionally substituted and/or optionally branched, C₄ to C₂₀ polyalkenyl; an optionally substituted and/or optionally branched C₂ to C₂₀ alkynyl, or an optionally substituted and/or optionally branched C₄ to C₂₀ polyalkynyl; optionally, a OCH₃ group can cyclize with a neighboring OH group to form a methylene dioxy bridge; or a derivative or analogue thereof.

In another embodiment, the first compound has the general formula I,

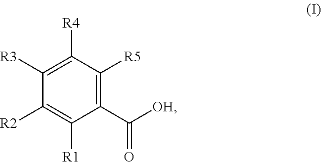

wherein:
R1 and R3 are each independently OH; OCH₃; O-aliphatic saturated or unsaturated acyl, O-glycoside; acylated O-glycoside; sulfated O-glycoside; or a sulfate.
R2 is CH₃, OH; O-glycoside; C-glycoside; sulfated O-glycoside; sulfated C-glycoside; a chlorine; a CHO (aldehyde); a sulfate; an optionally substituted and/or optionally branched, C₂ to C₁₅ alkenyl; or an optionally substituted and/or optionally branched, C₄ to C₁₅ polyalkenyl;
R4 is H; CH₃, OH; OCH₃; a chlorine, or a 3,3-dimethylallyl chain (1h);
R5 is H; an optionally substituted and/or optionally branched C₁ to C₂₀ alkyl; an optionally substituted and/or optionally branched, C₂ to C₂₀ alkenyl; or an optionally substituted and/or optionally branched, C₄ to C₂₀ polyalkenyl; optionally, a OCH₃ group can cyclize with a neighboring OH group to form a methylene dioxy bridge; or a derivative or analogue thereof.

In one embodiment, the first compound has the general formula I,

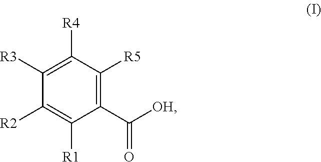

wherein:
R1 and R3 are each independently OH; OCH₃; O-aliphatic saturated or unsaturated acyl, O-glycoside; acylated O-glycoside; sulfated O-glycoside; or a sulfate.
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a substituted alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a); optionally a OCH₃ group can cyclize with a neighboring OH group to form a methylene dioxy bridge; or a derivative or analogue thereof.

In another embodiment, the first compound has the general formula I,

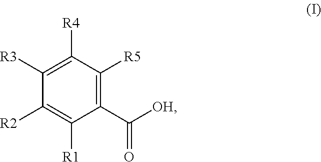

wherein:
R1 and R3 are each independently OH; OCH₃; O-aliphatic saturated or unsaturated acyl, O-glycoside; acylated O-glycoside; sulfated O-glycoside; or a sulfate.
R2 is OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a substituted alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a substituted alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
R4 is H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
R5 is H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a substituted alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a); optionally, a OCH3 group can cyclize with a neighboring OH group to form a methylene dioxy bridge; or a derivative or analogue thereof.

In another embodiment, the first compound has the general formula I,

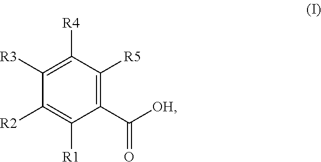

wherein:
R1 and R3 are each independently OH; OCH₃; O-aliphatic saturated or unsaturated acyl, O-glycoside; acylated O-glycoside; sulfated O-glycoside; or a sulfate.
R2 is CH₃, OH; O-glycoside; C-glycoside; sulfated O-glycoside; sulfated C-glycoside; a chlorine; a CHO (aldehyde); a sulfate; a C₅ isoprenoid chain selected from the group consisting of: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C10 isoprenoid chain selected from the group consisting of: 3,7-Dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: (2E,6E)-3,7,11-Trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); or a (substituted) polyalkenyl chain selected from the group consisting of: 8-(3,4-dihydroxyphenyl)-4,7-octadien-1-yl (18a);
R4 is H; CH3, OH; OCH₃; a chlorine, or a 3,3-dimethylallyl chain (1h);
R5 is H; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (in), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain among the following representatives: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a); optionally, a OCH₃ group can cyclize with a neighboring OH group to form a methylene dioxy bridge; or a derivative or analogue thereof.

In one embodiment, the first compound has the general formula I,

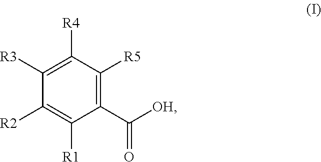

wherein
R2 is hydrogen and R4 is not hydrogen;
or a derivative or analogue thereof.

In one embodiment, the first compound has the general formula I,

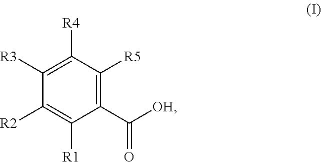

wherein:
R2 is hydrogen and R5 is not hydrogen;
or a derivative or analogue thereof.

In one embodiment, the first compound has the general formula I,

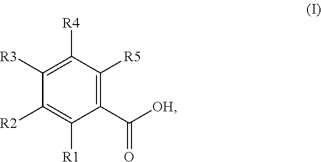

wherein:
R1 and R3 are each independently OH; OCH₃; O-glycoside; or a sulfate;
R2 is H; 3-methyl-2-buten-1-yl (3,3-dimethylallyl) (1h); or (2E)-3,7-dimethyl-2,6-octadien-1-yl (geranyl) (2h);
R4 is H; and
R5 is pentyl; benzyl (6a); 2-phenethyl (6b);
or phenylethenyl (14a), or a derivative or analogue thereof.

In one embodiment, the first compound has the general formula I,

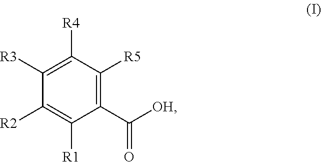

wherein:
R1=OH; O-glycoside; or a sulfate;
R2 is H; [3-methyl-3-(4-methyl-3-penten-1-yl)-2-oxiranyl]methyl (geranyl 2,3-epoxide) (2e); 3-methyl-6-(1-methylethenyl)-2-cyclohexen-1-yl (2g); (2E)-3,7-dimethyl-2,6-octadien-1-yl (geranyl) (2h); (2Z)-3,7-dimethyl-2,6-octadien-1-yl (neryl) (2i); or 5-methyl-2-(1-methylethyl)cyclohexyl (2o);
R3 is OH; OCH₃; O-glycoside; or a sulfate;
R4 is H;
R5 is CH3; n-propyl, n-butyl, or pentyl, or a derivative or analogue thereof.

In one embodiment, the first compound is of the general Formula I is cannabidiolic acid (CAS number 1244-58-2) or a derivative or analogue thereof.

In one embodiment, the first compound is CBDA, CBGA, CBNA, or a derivative or analogue thereof.

In one embodiment, the first compound is CBGA, CBNA, or a derivative or analogue thereof.

In one embodiment, the first compound is CBGA or a derivative or analogue thereof.

In one embodiment, the first compound is CBNA or a derivative or analogue thereof.

In one embodiment, the composition for use in the activation of AMPK comprises the first compound of cannabidiolic acid (CAS number 1244-58-2) or a derivative or analogue thereof as an AMP-activated protein kinase (AMPK) activator and the second compound of a sodium-glucose cotransporter-2 (SGLT2) inhibitor or its derivatives or analogues thereof.

In one embodiment, the composition for use in the activation of AMPK comprises the first compound of cannabidiolic acid (CAS number 1244-58-2), CBGA, CBNA, or a derivative or analogue thereof as an AMP-activated protein kinase (AMPK) activator and the second compound selected from the group consisting of dapagliflozin, canagliflozin, empagliflozin, artigliflozin, resmogliflozin, sergliflozin, phlorizin and their derivatives as a sodium-glucose cotransporter-2 (SGLT2) inhibitor.

In one embodiment, the composition for use in the activation of AMPK comprises the first compound of cannabidiolic acid (CAS number 1244-58-2), CBGA, CBNA, or a derivative or analogue thereof as an AMP-activated protein kinase (AMPK) activator and the second compound selected from the group consisting of dapagliflozin, canagliflozin, phlorizin and empagliflozin and their derivatives as a sodium-glucose cotransporter-2 (SGLT2) inhibitor.

In one embodiment, the composition for use in the activation of AMPK comprises the first compound of cannabidiolic acid (CAS number 1244-58-2), CBGA, CBNA, or a derivative or analogue thereof as an AMP-activated protein kinase (AMPK) activator and the second compound comprising at least one of dapagliflozin, canagliflozin, empagliflozin, artigliflozin, resmogliflozin, sergliflozin, phlorizin and their derivatives as a sodium-glucose cotransporter-2 (SGLT2) inhibitor.

In one embodiment, the composition for use in the activation of AMPK comprises the first compound of cannabidiolic acid (CAS number 1244-58-2), CBGA, CBNA, or a derivative or analogue thereof as an AMP-activated protein kinase (AMPK) activator and the second compound comprising dapagliflozin or its derivatives as a sodium-glucose cotransporter-2 (SGLT2) inhibitor.

In one preferred embodiment, the composition for use in the activation of AMPK comprises the first compound of cannabidiolic acid (CAS number 1244-58-2), CBGA, CBNA, or a derivative or analogue thereof as an AMP-activated protein kinase (AMPK) activator and the second compound comprising dapagliflozin or its derivatives or analogues as a sodium-glucose cotransporter-2 (SGLT2) inhibitor.

The first compound (e.g., an AMPK activator such as cannabidiolic acid (CAS number 1244-58-2), CBGA, CBNA, or a derivative or analogue thereof) and the second compound (e.g., a SGLT2 inhibitor such as dapagliflozin, phlorizin or its derivatives or analogue) of the composition for use in the activation of AMPK may be administered in accordance with the present invention in any pharmaceutically effective amount. Typically, a pharmaceutically effective amount will depend on the type, age, size, health status, lifestyle and/or genetic heritage of the subject. The pharmaceutically effective amount may be split into several smaller amounts and administered throughout the day so as the total daily intake is the effective amount. A person skilled in the art will be able to propose appropriate amounts of the first compound (e.g., an AMPK activator such as cannabidiolic acid (CAS number 1244-58-2), CBGA, CBNA, or a derivative or analogue thereof) and the second compound (e.g., a SGLT2 inhibitor such as dapagliflozin or its derivatives or analogue) to be consumed per day.

In one embodiment, the composition of the invention can have an acute effect that can be seen in less than one month. Additionally or alternatively, the composition can have a longterm effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day. In one embodiment, a single dose is not less than about 100 mg. In one embodiment, a single dose is not more than about 1000 mg. In one embodiment, a single dose is between about 100 mg and about 1000 mg.

In one embodiment, the first compound (e.g., an AMPK activator such as cannabidiolic acid (CAS number 1244-58-2), CBGA, CBNA, or a derivative or analogue thereof) in the present invention may be provided in an amount of between about 1 mg and about 10000 mg per daily dose, 5 mg and about 5000 mg per daily dose, about 10 mg and about 2000 mg per daily dose, about 50 mg and about 1500 mg per daily dose, between about 100 mg and about 1000 mg per daily dose, between about 150 mg and about 900 mg per daily dose, between about 200 mg and about 800 mg per daily dose, between about 250 mg and about 750 mg per daily dose, between about 300 mg and about 700 mg per daily dose, between about 350 mg and about 650 mg per daily dose, between about 400 mg and about 600 mg per daily dose, between about 450 mg and about 550 mg per daily dose, preferably about 500 mg per daily dose.

In one embodiment, the second compound (e.g., a SGLT2 inhibitor such as dapagliflozin, phlorizin or its derivatives or analogue) in the present invention is provided in an amount of between about 1 mg and about 2000 mg per daily dose, between about 50 mg and about 1500 mg per daily dose, or between about 100 mg and about 1000 mg per daily dose.

In one embodiment, the second compound (e.g., a SGLT2 inhibitor such as dapagliflozin or its derivatives or analogue) in the present invention is provided in an amount of between about 1.1 mg and about 40 mg per daily dose, between about 1.4 mg and about 38.8 mg per daily dose, between about 1.7 mg and about 37.6 mg per daily dose, between about 2.0 mg and about 36.4 mg per daily dose, between about 2.3 mg and about 35.2 mg per daily dose, between about 2.6 mg and about 34 mg per daily dose, between about 2.9 mg and about 32.8 mg per daily dose, between about 3.2 mg and about 31.6 mg per daily dose, between about 3.5 mg and about 30.4 mg per daily dose, between about 3.8 mg and about 29.2 mg per daily dose, between about 4.1 mg and about 28 mg per daily dose, between about 4.4 mg and about 26.8 mg per daily dose, between about 4.7 mg and about 25.6 mg per daily dose, between about 5.0 mg and about 24.4 mg per daily dose, between about 5.3 mg and about 23.2 mg per daily dose, between about 5.8 mg and about 22 mg per daily dose, between about 6.1 mg and about 20.8 mg per daily dose, between about 6.4 mg and about 19.6 mg per daily dose, between about 6.7 mg and about 18.4 mg per daily dose, between about 7.0 mg and about 17.2 mg per daily dose, between about 7.3 mg and about 16 mg per daily dose, between about 7.6 mg and about 14.8 mg per daily dose, between about 7.9 mg and about 13.6 mg per daily dose, between about 8.2 mg and about 12.4 mg per daily dose, between about 8.5 mg and about 11.2 mg per daily dose, between about 8.8 mg and about 6 mg per daily dose, between about 9.1 mg and about 11.0 mg per daily dose, between about 9.4 mg and about 10.8 mg per daily dose, between about 9.7 mg and about 10.4 mg per daily dose, preferably about 10 mg per daily dose.

In one embodiment, the composition for use in the activation of AMPK comprising both the first compound (e.g., an AMPK activator such as cannabidiolic acid (CAS number 1244-58-2), CBGA, CBNA, or a derivative or analogue thereof) in an amount of between about 100 mg and about 1000 mg per daily dose, preferably about 500 mg per daily dose and the second compound (e.g., a SGLT2 inhibitor such as dapagliflozin or its derivatives or analogue) in an amount of between about 1.1 mg and about 40 mg per daily dose, preferably about 10 mg per daily dose. In one preferred embodiment, the nutritional composition or nutritional supplement comprising both the first compound (e.g., an AMPK activator such as cannabidiolic acid (CAS number 1244-58-2), CBGA, CBNA, or a derivative or analogue thereof) in an amount of about 100 mg-1000 mg per daily dose and the second compound (e.g., a SGLT2 inhibitor such as dapagliflozin or its derivatives or analogue) in an amount of about 10 mg per daily dose.

In another embodiment, the nutritional composition or nutritional supplement comprising both the first compound (e.g., an AMPK activator such as cannabidiolic acid (CAS number 1244-58-2), CBGA, CBNA, or a derivative or analogue thereof) in an amount of about 100 mg per daily dose and the second compound (e.g., a SGLT2 inhibitor such as dapagliflozin or its derivatives or analogue) in an amount of about 10 mg per daily dose.

In another embodiment, the nutritional composition or nutritional supplement comprising both the first compound (e.g., an AMPK activator such as cannabidiolic acid (CAS number 1244-58-2), CBGA, CBNA, or a derivative or analogue thereof) in an amount of about 200 mg per daily dose and the second compound (e.g., a SGLT2 inhibitor such as dapagliflozin or its derivatives or analogue) in an amount of about 10 mg per daily dose.

In another embodiment, the nutritional composition or nutritional supplement comprising both the first compound (e.g., an AMPK activator such as cannabidiolic acid (CAS number 1244-58-2), CBGA, CBNA, or a derivative or analogue thereof) in an amount of about 300 mg per daily dose and the second compound (e.g., a SGLT2 inhibitor such as dapagliflozin or its derivatives or analogue) in an amount of about 10 mg per daily dose.

In another embodiment, the nutritional composition or nutritional supplement comprising both the first compound (e.g., an AMPK activator such as cannabidiolic acid (CAS number 1244-58-2), CBGA, CBNA, or a derivative or analogue thereof) in an amount of about 400 mg per daily dose and the second compound (e.g., a SGLT2 inhibitor such as dapagliflozin or its derivatives or analogue) in an amount of about 10 mg per daily dose.

In one embodiment, the composition comprising the first compound of cannabidiolic acid (CAS number 1244-58-2), CBGA, CBNA, or a derivative or analogue thereof as an AMP-activated protein kinase (AMPK) activator and the second compound comprising dapagliflozin or its derivatives as a sodium-glucose cotransporter-2 (SGLT2) inhibitor may be used as a nutritional composition or nutritional supplement. For example, the composition comprising the first compound (e.g., cannabidiolic acid (CAS number 1244-58-2), CBGA, CBNA, or a derivative or analogue thereof or a derivative or analogue thereof) and the second compound (e.g., dapagliflozin or its derivatives) might be beneficial to a human subject (such as an old adult or a patient whose metabolic health needs improvements) for synergistically activating AMPK, thus improving his/her metabolic health.

In one embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may be used for daily supplemental or routine administration. For example, the composition comprising the first compound (e.g., cannabidiolic acid (CAS number 1244-58-2), CBGA, CBNA, or a derivative or analogue thereof) and the second compound (e.g., dapagliflozin or its derivatives) may be administered to an old adult or a patient whose metabolic health needs improvements. In one embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may be administered to a human subject with a metabolic disorder such as obesity, type 2 diabetes, cardiovascular disease or with an obesity related disorder such as diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise at least one of an excipient, a diluent, or a carrier. In one embodiment, the first compound (e.g., cannabidiolic acid (CAS number 1244-58-2), CBGA, CBNA, or a derivative or analogue thereof) and the second compound (e.g., dapagliflozin or its derivatives) may be dissolved or suspended in a diluent or carrier.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise an excipient. An exemplary excipient may be an excipient described in the Handbook of Pharmaceutical Excipients, American Pharmaceutical Association (1986).

Non-limiting examples of suitable excipients may include a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient may be a buffering agent. Non-limiting examples of suitable buffering agents may include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments, an excipient may comprise a preservative. Non-limiting examples of suitable preservatives may include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In one embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise a binder. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In one embodiment, the binder may be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise a lubricant as an excipient. Non-limiting examples of suitable lubricants may include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical composition can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulfates (such as sodium lauryl sulfate, magnesium lauryl sulfate), sodium chloride, sodium benzoate, sodium acetate and talc or a combination thereof.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise a dispersion enhancer as an excipient. Non-limiting examples of suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may further comprise a disintegrant as an excipient. In some embodiments a disintegrant can be a non-effervescent disintegrant. Non-limiting examples of suitable non-effervescent disintegrants may include starches such as corns tarch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments, a disintegrant may be an effervescent disintegrant. Non-limiting examples of suitable effervescent disintegrants may include sodium bicarbonate in combination with citric acid, and sodium bicarbonate in combination with tartaric acid.

In some embodiments, an excipient may comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments, a flavoring agent may be selected from the group consisting of cinnamon oils; oil of wintergreen; peppermint oils; clover oil; hay oil; anise oil; Eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiment, an excipient may comprise a sweetener. Non-limiting examples of suitable sweeteners may include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may comprise a coloring agent. Non-limiting examples of suitable color agents may include food, drug and cosmetic colors (FD&C), drug and cosmetic colors (D&C), and external drug and cosmetic colors (Ext. D&C). A coloring agent may be used as dyes or their corresponding lakes.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may comprise a diluent. Non-limiting examples of diluents can include water, glycerol, methanol, ethanol, and other similar biocompatible diluents. In some embodiments, a diluent may be an aqueous acid such as acetic acid, citric acid, maleic acid, hydrochloric acid, phosphoric acid, nitric acid, sulfuric acid, or similar. In some embodiments, a diluent may be used to titrate a pH of a compound to a pH such as physiological pH to produce a salt as described above. In other cases, a diluent may be selected from a group comprising alkaline metal carbonates such as calcium carbonate; alkaline metal phosphates such as calcium phosphate; alkaline metal sulfates such as calcium sulfate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some embodiments, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may comprise a surfactant. Surfactants may be selected from, but not limited to, polyoxyethylene sorbitan fatty acid esters (polysorbates), sodium lauryl sulfate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

The composition may be administered by any method appreciated by the skilled artisan. For example, the first compound (e.g., cannabidiolic acid (CAS number 1244-58-2), CBGA, CBNA, or a derivative or analogue thereof) and the second compound (e.g., dapagliflozin, phlorizin or its derivatives) of the invention or composition thereof is preferably administered by oral administration. In some embodiments, the first compound (e.g., cannabidiolic acid (CAS number 1244-58-2), CBGA, CBNA, or a derivative or analogue thereof) and the second compound (e.g., dapagliflozin, phlorizin or its derivatives) of the invention or composition thereof may be administered by intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

In one embodiment, the composition of the invention may have an acute effect that can be seen in less than one month. Additionally or alternatively, the composition can have a long-term effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day. In one embodiment, a single dose is not less than about 100 mg. In one embodiment, a single dose is not more than about 1000 mg. In one embodiment, a single dose is between about 100 mg and about 1000 mg.

In one embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may be in a liquid form or in a solid form (e.g., solid dosage forms).

In some embodiments, solid dosage forms of the composition for oral administration may include capsules, tablets, caplets, pills, troches, lozenges, powders, and granules. In one embodiment, the first compound (e.g., cannabidiolic acid (CAS number 1244-58-2), CBGA, CBNA, or a derivative or analogue thereof) and the second compound (e.g., dapagliflozin, phlorizin or its derivatives) of the composition are in two separate solid dosage forms.

In one embodiment, the first compound (e.g., cannabidiolic acid (CAS number 1244-58-2), CBGA, CBNA, or a derivative or analogue thereof) and the second compound (e.g., dapagliflozin, phlorizin or its derivatives) of the composition are in the same solid dosage form.

For example, a capsule may comprise a core material comprising the composition comprising the first compound (e.g., cannabidiolic acid (CAS number 1244-58-2), CBGA, CBNA, or a derivative or analogue thereof) and/or the second compound (e.g., dapagliflozin, phlorizin or its derivatives) and a shell wall that encapsulates a core material. In some embodiments, a core material may comprise at least one of a solid, a liquid, and an emulsion. In some embodiments, a shell wall material may comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments, at least one polymer may function as taste-masking agents.

In some embodiments, tablets, pills, and the like may be compressed, multiply compressed, multiply layered, and/or coated. For example, a coating may be single or multiple. In some embodiments, a coating material (i.e., comprising the composition comprising the first compound (e.g., cannabidiolic acid (CAS number 1244-58-2), CBGA, CBNA, or a derivative or analogue thereof) and/or the second compound (e.g., dapagliflozin, phlorizin or its derivatives)) may comprise at least one of a saccharide, a polysaccharide, and glycoproteins extracted from at least one of a plant, a fungus, and a microbe. Non-limiting examples may include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments, a coating material may comprise a protein. In some embodiments, a coating material may comprise at least one of a fat and/or an oil. In some embodiments, the at least one of a fat and/or an oil may be high temperature melting. In some embodiments, the at least one of a fat and/or an oil may be hydrogenated or partially hydrogenated. In some embodiments, the at least one of a fat and/or an oil may be derived from a plant. In some embodiments, the at least one of a fat and/or an oil may comprise at least one of glycerides, free fatty acids, and fatty acid esters. In some embodiments, a coating material may comprise at least one edible wax. An edible wax may be derived from animals, insects, or plants. Non-limiting examples can include beeswax, lanolin, bayberry wax, carnauba wax, and rice bran wax. Tablets and pills can additionally be prepared with enteric coatings.

Liquid formulations of the composition comprising the first compound (e.g., cannabidiolic acid (CAS number 1244-58-2), CBGA, CBNA, or a derivative or analogue thereof) and/or the second compound (e.g., dapagliflozin, phlorizin or its derivatives) may include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some instances, a combination of various formulations may be administered. In some embodiments, a tablet, pill, and the like can be formulated for an extended release profile. In some embodiments, a composition may be formulated to increase the shelf stability when stored in a closed container under standard ambient conditions.

Each of the components in the composition of the present invention may be used in any amount that is effective in achieving the objective of the present invention (i.e., increasing breastmilk micronutrient levels of a subject after the subject gives birth). For example, the skilled artisan would be able to determine appropriate dosages depending on age, size and health status of each specific subject, on her lifestyle, as well as on her genetic heritage.

In one embodiment, the amounts used in the present application are amounts per daily dose. The amount of each component may be used as disclosed or changed (e.g., increased or decreased) depending on age, size and health status of each specific subject, on her lifestyle, as well as on her genetic heritage. In one embodiment, the nutritional composition or nutritional supplement of the present invention may be administered regularly, for example two times a day, daily, every two days or weekly.

In an aspect, the composition of the present invention may be in any form that is suitable to administer all the ingredients. For example, the composition of the present invention can be in the form of a powdered nutritional composition to be reconstituted in milk or water, a food product, a drink, a nutritional supplement or a nutraceutical.

When the composition of the present invention is in the form of a powdered nutritional composition to be reconstituted in milk or water, the nutritional composition or nutritional supplement may preferably comprise a protein source, a carbohydrate source and a lipid source, preferably together with lecithin. The composition may also comprise soya lecithin and/or a bulking agent. The protein source may be dried milk or dried skimmed milk. As carbohydrate source sucrose and/or maltodextrin may be used. The lipid source may be vegetable oil. The formulation may also alternatively or additionally contain glucose syrup, milk fat, magnesium citrate, choline salts and esters, prebiotic fibers, and/or ascorbyl palmitate. Flavor compounds, such as cocoa powder or honey, for example, may be added to provide taste variations.

In another aspect, the composition of the present invention may be a product selected from the group consisting of a nutritional product, a functional food product, a healthy ageing product, a dairy product, a dairy alternative product, a beverage product, a diet product, and a pet food product.

In one embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may be used for treating or preventing a condition, disorder, or disease related to type 2 diabetes, non-alcoholic fatty liver disease and/or obesity in a subject.

In one embodiment, the condition may be a metabolic disorder such as obesity, type 2 diabetes, cardiovascular disease or an obesity related disorder such as diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In another embodiment, the composition comprising the first compound (e.g., cannabidiolic acid (CAS number 1244-58-2), CBGA, CBNA, or a derivative or analogue thereof) and the second compound (e.g., dapagliflozin, phlorizin or its derivatives) may be used for treating or preventing a condition selected from the group consisting of obesity, type 2 diabetes, cardiovascular disease, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

### Methods

Another aspect of the present disclosure is a method for improving metabolic health (e.g., treating or preventing a metabolic disorder or an obesity related disorder) by synergistically activating AMPK. In one embodiment, the method comprises administering a subject in need thereof a composition comprising a combination of an AMP-activated protein kinase (AMPK) activator (e.g., CBDA) and a sodium-glucose cotransporter-2 (SGLT2) inhibitor.

In one embodiment, the method for improving metabolic health comprises treating or preventing a metabolic disorder. In one embodiment, the metabolic disorder comprises obesity, type 2 diabetes, cardiovascular disease. In one embodiment, the metabolic disorder is selected from the group consisting of obesity, type 2 diabetes, and cardiovascular disease.

In another embodiment, the method for improving metabolic health comprises treating or preventing an obesity related disorder. In one embodiment, the obesity related disorder comprises diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. In one embodiment, the obesity related disorder is selected from the group consisting of diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In one embodiment, the present disclosure is a method of treating or preventing a condition selected from the group consisting of obesity, type 2 diabetes, cardiovascular disease, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia.

In another embodiment, the present disclosure is a method of treating or preventing a condition, disorder, or disease related to type 2 diabetes, non-alcoholic fatty liver disease and/or obesity in a subject by activating AMPK.

As disclosed above, the method comprises administering a subject in need thereof a composition comprising a combination of an AMP-activated protein kinase (AMPK) activator (e.g., CBDA) and a sodium-glucose cotransporter-2 (SGLT2) inhibitor to activate AMPK.

In one embodiment, the first compound of the general Formula I is cannabidiolic acid, CAS number 1244-58-2, and the second compound is selected from the group consisting of empagliflozin, canagliflozin, dapagliflozin, phlorizin and ertugliflozin.

In one embodiment, the first compound is CBDA, CBGA or CBNA, and the second compound is selected from the group consisting of empagliflozin, canagliflozin, dapagliflozin, phlorizin and ertugliflozin.

In one embodiment, the second compound is selected from the group consisting of empagliflozin, canagliflozin and dapagliflozin.

In one embodiment, the second compound comprises dapagliflozin.

In one embodiment, the second compound is dapagliflozin.

In one embodiment, the second compound is phlorizin.

In one embodiment, the first compound is CBDA, CBGA or CBNA, and the second compound is phlorizin.

In one embodiment, the first compound of the general Formula I is cannabidiolic acid, CAS number 1244-58-2, and the second compound is dapagliflozin.

In one embodiment, the method comprises administering a subject in need thereof a composition comprising a combination of the first compound of cannabidiolic acid (CAS number 1244-58-2) and the second compound of dapagliflozin to activate AMPK.

In one embodiment, the activation of AMPK is through either a direct activation mechanism or an indirect activation mechanism.

In one embodiment, the activation of AMPK is through a direct activation mechanism.

In one embodiment, the activation of AMPK occurs at any tissue in the subject related to the condition, disorder, or disease.

In one embodiment, the activation of AMPK is in muscle, liver and/or kidney tissues.

In one embodiment, the activation of AMPK is at activated AMPK which comprises an α2 subunit, a β1 subunit, and a γ1 subunit.

In one embodiment, the first compound is obtained from a plant or plant extract.

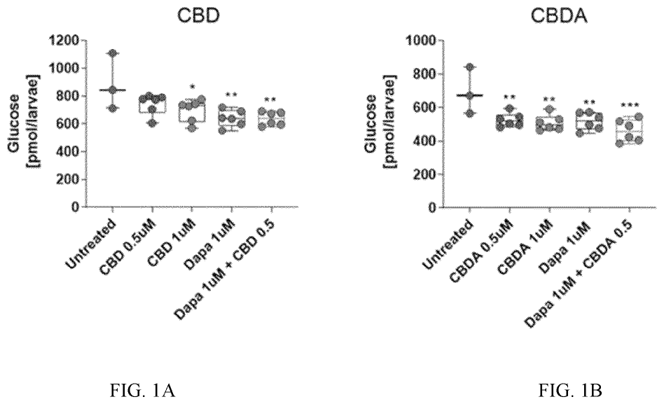

As shown in Example 1 and FIG. 1, the subjects were administered with the composition comprising both CBD or CBDA about 1 μM or about 0.5 μM and Dapa about 1 μM. Example 1, FIG. 1A shows that a combination of CBD and Dapa (a SGLT-2 inhibitor) improves the glucose clearance of Zebrafish as compared with CBD and Dapa alone. FIG. 1B shows that a combination of CBDA and Dapa (an SGLT-2 inhibitor) improves the glucose clearance of Zebrafish as compared with CBDA and Dapa alone.

In one embodiment, the composition of the present invention can lead to at least 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% increase on glucose clearance in the subject as compared with the control subjects who were not administered with the composition comprising the first compound of CBD or CBDA or the second compound of Dapa of the present invention or as compared with the control subjects who were administered with the composition comprising either the first compound of CBD or CBDA or the second compound of Dapa.

In one embodiment, the increase on glucose clearance can be sustained up to 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, or 48 months.

### EXAMPLES

### Example 1

FIG. 1 (FIG. 1A and FIG. 1B) is a set of graphs showing glucose clearance in Zebrafish according to certain embodiments of the present invention. FIG. 1A shows that a combination of CBD and Dapa (a SGLT-2 inhibitor) improves the glucose clearance of Zebrafish as compared with CBD and Dapa alone. FIG. 1B shows that a combination of CBDA and Dapa (an SGLT-2 inhibitor) improves the glucose clearance of Zebrafish as compared with CBDA and Dapa alone.

1. A method for activation of AMPK comprising administering the composition to an individual in need of same, the composition comprising:
a first compound; and
a second compound;
wherein the first compound has the general formula I, 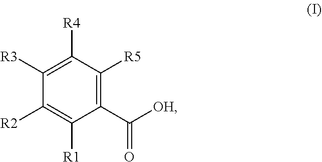
wherein R1 and R3 are each independently selected from the group consisting of OH; OCH₃; O-aliphatic saturated or unsaturated acyl, O-glycoside; acylated O-glycoside; sulfated O-glycoside; and a sulfate;
R2 is selected from the group consisting of OH; OCH₃; O-glycoside; C-glycoside;
acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and/or branched C₁ to C₂₀ alkyl; substituted and/or branched, C₂ to C₂₀ alkenyl; substituted and branched, C₄ to C₂₀ polyalkenyl; substituted and branched C₂ to C₂₀ alkynyl, and substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl; a OCH₃ group can cyclize with a neighboring OH group to form a methylene dioxy bridge;
or a derivative or analogue thereof; or
the first compound is cannabigerolic acid (CBGA) or cannabinolic acid (CBNA); and
wherein the second compound is a sodium-glucose cotransporter-2 (SGLT2) inhibitor.

2. The composition of claim 1, wherein the sodium-glucose cotransporter-2 (SGLT2) inhibitor is selected from the group consisting of empagliflozin, canagliflozin, dapagliflozin, ertugliflozin, phlorizin and mixtures thereof.

3. The composition of claim 1, wherein the sodium-glucose cotransporter-2 (SGLT2) inhibitor is selected from the group consisting of empagliflozin, canagliflozin, dapagliflozin, and mixtures thereof.

4. The composition of claim 1, wherein the sodium-glucose cotransporter-2 (SGLT2) inhibitor comprises dapagliflozin.

5. The composition of claim 1, wherein the sodium-glucose cotransporter-2 (SGLT2) inhibitor is dapagliflozin.

6. The composition of claim 1, wherein the first compound has the general formula I, 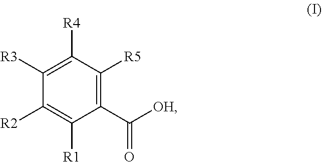
wherein:
R1 and R3 are each independently selected from the group consisting of OH; OCH₃; O-aliphatic saturated or unsaturated acyl, O-glycoside; acylated O-glycoside; or sulfated O-glycoside;
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; or a sulfonate; substituted and branched C₁ to C₂₀ alkyl; substituted and branched, C₂ to C₂₀ alkenyl; substituted and branched, C₄ to C₁₀ polyalkenyl; substituted and branched C₂ to C₂₀ alkynyl, substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; or a sulfonate; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; or a sulfonate; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl; a OCH₃ group can cyclize with a neighboring OH group to form a methylene dioxy bridge;
or a derivative or analogue thereof.

7. The composition of claim 1, wherein the first compound has the general formula I, 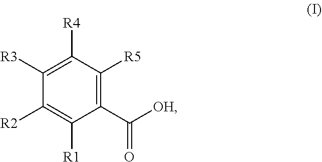
wherein:
R1 and R3 are each independently selected from the group consisting of OH; OCH₃; O-aliphatic saturated or unsaturated acyl, O-glycoside; and acylated O-glycoside;
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; or a nitro; an optionally substituted and/or optionally branched C₁ to C₂₀ alkyl; substituted and branched, C₁₀ to C₂₀ alkenyl; substituted and branched, C₁₀ to C₂₀ polyalkenyl; substituted and branched C₂ to C₂₀ alkynyl, substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; or a nitro; an optionally substituted and/or optionally branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; or a nitro; branched C₁ to C₂₀ alkyl; branched, C₂ to C₂₀ alkenyl; branched, C₄ to C₂₀ polyalkenyl; branched C₂ to C₂₀ alkynyl, branched C₄ to C₂₀ polyalkynyl; a OCH₃ group can cyclize with a neighboring OH group to form a methylene dioxy bridge;
and a derivative or analogue thereof.

8. The composition of claim 1, wherein the first compound has the general formula I, 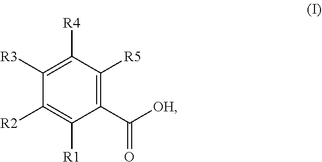
wherein:
R1 and R3 are each independently selected from the group consisting of OH; OCH₃; O-aliphatic saturated or unsaturated acyl, O-glycoside; or acylated O-glycoside;
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; or an ester; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; or an ester; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; or an ester; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl; optionally, a OCH₃ group can cyclize with a neighboring OH group to form a methylene dioxy bridge;
and a derivative or analogue thereof.

9. The composition of claim 1, wherein the first compound has the general formula I, 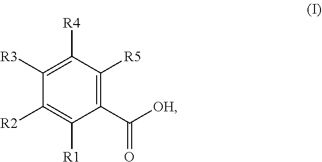
wherein:
R1 and R3 are each independently selected from the group consisting of OH; OCH₃; O-aliphatic saturated or unsaturated acyl, O-glycoside; acylated O-glycoside; sulfated O-glycoside; and a sulfate.
R2 is selected from the group consisting of CH₃, OH; O-glycoside; C-glycoside; sulfated O-glycoside; sulfated C-glycoside; a chlorine; a CHO (aldehyde); a sulfate; substituted and branched, C₂ to C₁₅ alkenyl; and branched, C₄ to C₁₅ polyalkenyl;
R4 is selected from the group consisting of H; CH₃, OH; OCH₃; a chlorine, or a 3,3-dimethylallyl chain (1h);
R5 is selected from the group consisting of H; substituted and branched C₁ to C₂₀ alkyl; substituted and branched, C₂ to C₂₀ alkenyl; substituted and branched, C₄ to C₂₀ polyalkenyl; a OCH₃ group can cyclize with a neighboring OH group to form a methylene dioxy bridge;
and a derivative or analogue thereof.

10. The composition of claim 1, wherein the first compound has the general formula I, 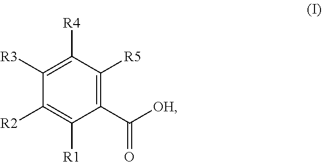
wherein:
R1 and R3 are each independently selected from the group consisting of OH; OCH₃; O-aliphatic saturated or unsaturated acyl, O-glycoside; acylated O-glycoside; sulfated O-glycoside; and a sulfate.
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a substituted alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a);
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), a 8-(3,4-dihydroxyphenyl)-4,7-octadien-1-yl (18a); optionally a OCH₃ group can cyclize with a neighboring OH group to form a methylene dioxy bridge;
or a derivative or analogue thereof.

11. The composition of claim 1, wherein the first compound has the general formula I, 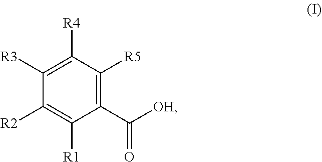
wherein:
R1 and R3 are each independently selected from the group consisting of OH; OCH₃; O-aliphatic saturated or unsaturated acyl, O-glycoside; acylated O-glycoside; sulfated O-glycoside; or a sulfate.
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a substituted alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a substituted alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a);
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a sulfate; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p); a C₁₀ isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a substituted alkenyl chain selected from the group consisting of: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a substituted polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a); optionally, a OCH3 group can cyclize with a neighboring OH group to form a methylene dioxy bridge;
or a derivative or analogue thereof.

12. The composition of claim 1, wherein the first compound has the general formula I, 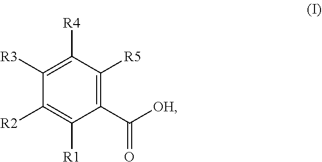
wherein:
R1 and R3 are each independently selected from the group consisting of OH; OCH₃; O-aliphatic saturated or unsaturated acyl, O-glycoside; acylated O-glycoside; sulfated O-glycoside; and a sulfate.
R2 is selected from the group consisting of CH₃, OH; O-glycoside; C-glycoside; sulfated O-glycoside; sulfated C-glycoside; a chlorine; a CHO (aldehyde); a sulfate; a C₅ isoprenoid chain selected from the group consisting of: 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p);
a C10 isoprenoid chain selected from the group consisting of: 3,7-Dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: (2E,6E)-3,7,11-Trimethyl-2,6,10-dodecatrien-1-yl (farnesyl) (3b), 7-hydroxy-3,7,11-trimethyl-2,10-dodecadien-1-yl (3c), 3-methyl-6-[5-(2-methyl-1-propen-1-yl)-3-furanyl]-2-hexen-1-yl (3d); and
a (substituted) polyalkenyl chain selected from the group consisting of: 8-(3,4-dihydroxyphenyl)-4,7-octadien-1-yl (18a);
R4 is selected from the group consisting of H; CH3, OH; OCH₃; a chlorine, or a 3,3-dimethylallyl chain (1h);
R5 is selected from the group consisting of H; a C₅ isoprenoid chain selected from the group consisting of: 3-Methylbutyl (1a), 4-hydroxy-3-methylbutyl (1b), 3-hydroxy-3-methylbutyl (1c), 2-hydroxy-3-methylbutyl (1d), (3,3-dimethyl-2-oxiranyl)methyl (epoxyprenyl) (1e), 2,3-dihydroxy-3-methylbutyl (1f), 3-methyl-2-oxobutyl (1g), 3-methyl-2-buten-1-yl (3,3-dimethylallyl) (1h), 1,1-dimethyl-2-propen-1-yl (1,1-dimethylallyl) (1i), 3-methyl-1-buten-1-yl (1j), 4-hydroxy-3-methyl-2-buten-1-yl (1k), 1-hydroxy-3-methyl-2-buten-1-yl (1l), 3-hydroxy-3-methyl-1-butenyl (1m), 4-hydroxy-3-methylbut-1-en-1-yl (1n), 2-hydroxy-3-methyl-3-buten-1-yl (1o), 3-methyl-1,3-butadienyl (1p);
a C10 isoprenoid chain selected from the group consisting of: 3,7-Dimethyloctyl (tetrahydrogeranyl) (2a), 8-hydroxy-3,7-dimethyloctyl (2b), 3,7-dimethyloct-6-en-1-yl (citronellyl) (2c), 6,7-dihydroxy-3,7-dimethyl-octa-2-enyl (2d), [3-methyl-3-(4-methyl-3-penten-1-yl)-2-oxiranyl]methyl (geranyl 2,3-epoxide) (2e), 5-hydroxy-5-methyl-2-(1-methylethenyl)hexyl (2f), 3-methyl-6-(1-methylethenyl)-2-cyclohexen-1-yl (2g), (2E)-3,7-dimethyl-2,6-octadien-1-yl (geranyl) (2h), (2Z)-3,7-dimethyl-2,6-octadien-1-yl (neryl) (2i), 5-methyl-2-(1-methylethenyl)-4-hexen-1-yl (lavandulyl) (2j), 5-hydroxy-3,7-dimethyl-2,6-octadienyl (2k), 6-hydroxy-3,7-dimethyl-2,7-octadien-1-yl (2l), 3,7-dimethyl-7-hydroxy-2,5-octadienyl (2m), 3,7-dimethyl-5-oxo-2,6-octadienyl (2n), 5-methyl-2-(1-methylethyl)cyclohexyl (2o); a C₁₅ isoprenoid chain selected from the group consisting of: 3,7,11-Trimethyldodecyl (hexahydrofarnesyl) (3a), (2E,6E)-3,7,11-trimethyl-2,6,10-dodecatrien-1-yl (farnesyl) (3b), 7-hydroxy-3,7,11-trimethyl-2,10-dodecadien-1-yl (3c), 3-methyl-6-[5-(2-methylprop-1-en-1-yl)furan-3-yl]hex-2-en-1-yl (3d); a C₂₀ isoprenoid chain selected from the group consisting of: 3,7,11,15-Tetramethylhexadecyl (phytanyl) (4a), (2E,6E,10E)-3,7,11,15-tetramethylhexadeca-2,6,10,14-tetraen-1-yl (geranylgeranyl) (4b); a (substituted) alkyl chain selected from the group consisting of: Methyl, ethyl, propyl, butyl, pentyl, heptyl, nonyl undecyl, pentadecyl, heptadecyl, eicosyl, hydroxymethyl (5a), 6-hydroxyheptyl (5b), 8-hydroxynonyl (5c), 12-hydroxytridecyl (5d), 2-hydroxytridecyl (5e), 2,12-dihydroxytridecyl (5f), 2-hydroxypentadecyl (5g), 14-hydroxypentadecyl (5h), 16-hydroxyheptadecyl (5i), 10-methoxyundecyl (5j), 12-methoxytridecyl (5k), 2-(acetyloxy)pentadecyl (5l), 2-(acetyloxy)-13-hydroxytridecyl (5m), 2-(acetyloxy)-12-hydroxytridecyl (5n), 2,12-bis(acetyloxy)tridecyl (5o), 2-(acetyloxy)tridecyl (5p), benzyl (6a), 2-phenylethyl (6b), 2-(4-hydroxyphenyl)ethyl (6c), 2-(3,4-dihydroxyphenyl)ethyl (6d), 2-(4-methoxyphenyl)ethyl (6e), 2-hydroxy-2-phenylyethyl (7a), 2-hydroxy-2-(2-hydroxyphenyl)ethyl (7b), tetrahydro-6-methyl-2H-pyran-2-yl)methyl (8a), 1,2-dioxopropyl (9a), 3-oxopentyl (9b), 2-oxopentyl (9c), 2-oxoheptyl (9d), 2-oxononyl (9e), 14-oxopentadecyl (9f), 2-oxotridecyl (9g), 1-hydroxy-2-oxopropyl (10a), 13-hydroxy-2-oxotridecyl (10b), 2-(4-hydroxyphenyl)-2-oxoethyl (11a), 2-oxo-2-phenylethyl (11b); a (substituted) alkenyl chain among the following representatives: ethenyl (12a), 1-propenyl (12b), 1-methylethenyl (12c), 1-methyl-1-propen-1-yl (12d), 8-pentadecen-1-yl (12e), 8-heptadecen-1-yl (12f), 10-heptadecen-1-yl (12g), 10-(acetyloxy)-8-pentadecenyl (13a), 2-phenylethenyl (14a), 1-hydroxymethylene-2-oxopropyl (15a); or a (substituted) polyalkenyl chain selected from the group consisting of: 8,11-heptadecadien-1-yl (16a), 1-oxo-2,4-octadien-1-yl (17a), 4,6-dihydroxy-6-methyl-3-oxo-1,4-cyclohexadien-1-yl (17b), (6-methyl-4-oxo-4H-pyran-2-yl)methyl (17c), 8-(3,4-dihydroxyphenyl)-4,7-octadien-1-yl (18a); optionally, a OCH₃ group can cyclize with a neighboring OH group to form a methylene dioxy bridge;
and a derivative or analogue thereof.

13. The composition of claim 6, wherein the first compound has the general formula I, 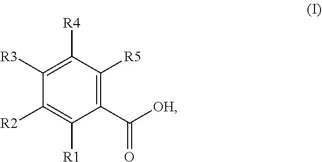
wherein
R2 is hydrogen and R4 is not hydrogen.

14. The composition of claim 6, wherein the first compound has the general formula I, 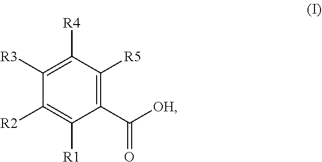
wherein:
R2 is hydrogen and R5 is not hydrogen;
or a derivative or analogue thereof.

15. The composition of claim 1, wherein the first compound has the general formula I, 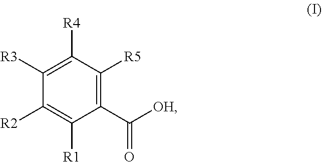
wherein:
R1 and R3 are each independently selected from the group consisting of OH; OCH₃; O-glycoside; and a sulfate;
R2 is selected from the group consisting of H; 3-methyl-2-buten-1-yl (3,3-dimethylallyl) (1h); and (2E)-3,7-dimethyl-2,6-octadien-1-yl (geranyl) (2h);
R4 is H; and
R5 is pentyl; benzyl (6a); 2-phenethyl (6b); and phenylethenyl (14a),
and a derivative or analogue thereof.

16. (canceled)

17. The composition of claim 1, wherein the first compound of the general Formula I is cannabidiolic acid, CAS number 1244-58-2.

18-28. (canceled)

29. An in vitro method of activating AMPK, the method comprising contacting the composition comprising:
a first compound; and
a second compound;
wherein the first compound has the general formula I, 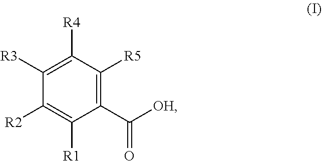
wherein R1 and R3 are each independently selected from the group consisting of OH; OCH₃; O-aliphatic saturated or unsaturated acyl, O-glycoside; acylated O-glycoside; sulfated O-glycoside; and a sulfate;
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and/or branched C₁ to C₂₀ alkyl; substituted and/or branched, C₂ to C₂₀ alkenyl; substituted and branched, C₄ to C₂₀ polyalkenyl; substituted and branched C₂ to C₂₀ alkynyl, and substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; substituted and branched C₁ to C₂₀ alkyl; substituted and branched, C₂ to C₂₀ alkenyl; substituted and branched, C₄ to C₂₀ polyalkenyl substituted and branched C₂ to C₂₀ alkynyl, substituted and branched C₄ to C₂₀ polyalkynyl a OCH₃ group can cyclize with a neighboring OH group to form a methylene dioxy bridge;
or a derivative or analogue thereof; or
the first compound is cannabigerolic acid (CBGA) or cannabinolic acid (CBNA); and
wherein the second compound is a sodium-glucose cotransporter-2 (SGLT2) inhibitor, with AMPK.

30. A method of treatment or prevention of a condition, disorder, or disease related to type 2 diabetes, non-alcoholic fatty liver disease and/or obesity, the method comprising administration of the composition comprising:
a first compound; and
a second compound;
wherein the first compound has the general formula I, 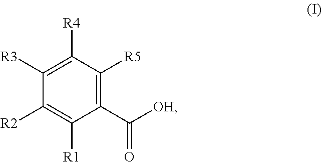
wherein R1 and R3 are each independently selected from the group consisting of OH; OCH₃; O-aliphatic saturated or unsaturated acyl, O-glycoside; acylated O-glycoside; sulfated O-glycoside; and a sulfate;
R2 is selected from the group consisting of OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a primary, secondary, or tertiary amine; a primary or secondary amide; a cyano; a nitro; a sulfonate; a sulfate; substituted and/or branched C₁ to C₂₀ alkyl; substituted and/or branched, C₂ to C₂₀ alkenyl; substituted and branched, C₄ to C₂₀ polyalkenyl; substituted and branched C₂ to C₂₀ alkynyl, and substituted and branched C₄ to C₂₀ polyalkynyl;
R4 is selected from the group consisting of H; OH; OCH₃; O-glycoside; C-glycoside; acylated O-glycoside; acylated C-glycoside; sulfated O-glycoside; sulfated C-glycoside; a Cl, I, or F halogen atom; a primary, secondary, or tertiary alcohol; a ketone; an aldehyde; an ester; a primary, secondary, or tertiary amine; a primary or secondary amide; a cyano; a nitro; a sulfonate; a sulfate; substituted and branched C1 to C20 alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl;
R5 is selected from the group consisting of H; OH; OCH₃; O-glycoside; C-glycoside; acylated O-glycoside; acylated C-glycoside; sulfated O-glycoside; sulfated C-glycoside; a halogen; a primary, secondary, or tertiary alcohol; a ketone; an aldehyde; an ester; a secondary, or tertiary amine; a primary or secondary amide; a cyano; a nitro; a sulfonate; a sulfate; substituted and branched C₁ to C₂₀ alkyl; substituted and branched, C₂ to C₂₀ alkenyl; substituted and branched, C₄ to C₂₀ polyalkenyl; substituted and branched C₂ to C₂₀ alkynyl, substituted and branched C₄ to C₂₀ polyalkynyl; a OCH₃ group can cyclize with a neighboring OH group to form a methylene dioxy bridge;
or a derivative or analogue thereof; or
the first compound is cannabigerolic acid (CBGA) or cannabinolic acid (CBNA); and
wherein the second compound is a sodium-glucose cotransporter-2 (SGLT2) inhibitor.

31. The method of claim 30, wherein the first compound of the general Formula I is selected from the group consisting of cannabidiolic acid, CAS number 1244-58-2, CBGA or CBNA and the second compound is selected from the group consisting of empagliflozin, canagliflozin, dapagliflozin, ertugliflozin, phlorizin and mixtures thereof.

32. The method of claim 31, wherein the second compound is selected from the group consisting of empagliflozin, canagliflozin, dapagliflozin, and mixtures thereof.

33-35. (canceled)
